(19) United States
(12) Patent Application Publication
(10) Pub. No.: US 2020/0063202 A1
(43) Pub. Date: Feb. 27, 2020

(54) METHODS OF USING GENETIC MARKERS ASSOCIATED WITH ENDOMETRIOSIS

(71) Applicant: Juneau Biosciences, L.L.C., Salt Lake City, UT (US)

(72) Inventors: Hans ALBERTSEN, Salt Lake City, UT (US); Rakesh N. CHETTIER, Salt Lake City, UT (US); Kenneth WARD, Salt Lake City, UT (US)

(21) Appl. No.: 16/395,087

(22) Filed: Apr. 25, 2019

Related U.S. Application Data
(60) Provisional application No. 62/662,469, filed on Apr. 25, 2018.

(51) Int. Cl. C12Q 1/6883; G16B 20/20; C12Q 1/6869; G16B 30/00; G16H 15/00

(52) U.S. Cl. CPC C12Q 1/6883; G16B 20/20; G16H 15/00; G16B 30/00; C12Q 1/6869

(57) ABSTRACT

Disclosed herein are methods of using genetic markers associated with endometriosis, for example via a computer-implemented program to predict risk of developing endometriosis, and methods of preventing or treating endometriosis or a symptom thereof. For example, the present disclosure provides a method of testing for endometriosis and treating a subject having at least one genetic mutation in at least one gene of UGT2B28, USP17L2 (alias DUBS), and METTL11B such that the subject is prevented from developing endometriosis or such that endometriosis in the subject is prevented from progressing. The treatment may be a surgical intervention, a hormone treatment, a pharmaceutical treating, or a combination thereof.

### CROSS REFERENCE

This application claims the benefit of U.S. Provisional Application No. 62/662,469, filed Apr. 25, 2018, which is incorporated herein by reference in its entirety.

### BRIEF SUMMARY

The inventive embodiments provided in this Brief Summary are meant to be illustrative only and to provide an overview of selective embodiments disclosed herein. The Brief Summary, being illustrative and selective, does not limit the scope of any claim, does not provide the entire scope of inventive embodiments disclosed or contemplated herein, and should not be construed as limiting or constraining the scope of this disclosure or any claimed inventive embodiment.

In some of many aspects, the present disclosure provides a method of testing for endometriosis and treating a patient having at least one genetic mutation in at least one gene of UGT2B28 (UDP glucuronosyltransferase family 2 member B28), USP17L2 (ubiquitin specific peptidase 17-like family member 2, as known as DUBS), and METTL11B (methyltransferase like 11B) such that the patient is prevented from developing endometriosis or such that endometriosis in the patient is prevented from progressing. The treatment may be a surgical intervention, a hormone treatment, a pharmaceutical treating, or a combination thereof.

In some aspects, provided herein is a method comprising assaying a genetic sample of a patient, detecting in said sample at least one genetic mutation in at least one gene of UGT2B28, USP17L2, and METTL11B, and applying at least one endometriosis therapeutic to said patient.

In some aspects, provided herein is a method that comprises applying at least one endometriosis therapeutic to a patient having at least one genetic mutation in at least one gene of UGT2B28, USP17L2, and METTTL11B in the DNA of said patient.

In some aspects, provided herein is a method that comprises: (a) hybridizing a nucleic acid probe to a nucleic acid sample from a human subject suspected of having or developing endometriosis; and (b) detecting a genetic variant in a panel comprising two or more genetic variants defining a minor allele listed in Tables 1 and 2.

In some aspects, provided herein is a method that comprises detecting one or more genetic variants defining a minor allele listed in Tables 1 and 2 in genetic material from a human subject suspected of having or developing endometriosis.

In some aspects, provided herein is a method that comprises: (a) sequencing all or a portion of one or more genes or gene expression products selected from the group consisting of UGT2B28, USP17L2, METTL11B and any combinations thereof to identify one or more protein damaging or loss of function variants in a human subject suspected of having or developing endometriosis; and (b) diagnosing the human subject as having or being at risk of developing when one or more protein damaging or loss of function variant is identified.

### INCORPORATION BY REFERENCE

All publications, patents, and patent applications mentioned, disclosed or referenced in this specification are herein incorporated by reference in their entirety and to the same extent as if each individual publication, patent, or patent application was specifically and individually indicated to be incorporated by reference.

### BRIEF DESCRIPTION OF THE DRAWINGS

FIG. 1 is a diagram showing pedigree of the studied Greek family. Partially filled circles represent women with endometriosis, open circles represent women without endometriosis, the circle with a diagonal line represents women of unknown phenotypic status, and open squares represent males. Diagonal lines represent individuals that were diseased at the time the pedigree was recorded. Case numbers 1-7 indicate the family members studied.

FIG. 2 is a diagram showing pedigree of the studied ESP148 family. Partially filled circles represent women with endometriosis, open circles represent women without endometriosis, the circle with a diagonal line represents women of unknown phenotypic status, and open squares represent males. Diagonal lines represent individuals that were diseased at the time the pedigree was recorded. Case numbers 1-8 indicate the family members studied.

FIG. 3 is a diagram showing a computer-based system that may be programmed or otherwise configured to implement methods provided herein.

FIG. 4 is a diagram showing a method and system as disclosed herein.

### DETAILED DESCRIPTION

Unless defined otherwise, all technical and scientific terms used herein have the same meaning as commonly understood by one of the ordinary skill in the art to which this invention belongs. Although any methods and materials similar or equivalent to those described herein can be used in the practice or testing of the compositions or unit doses herein, some methods and materials are now described. Unless mentioned otherwise, the techniques employed or contemplated herein are standard methodologies. The materials, methods and examples are illustrative only and not limiting.

The details of one or more inventive instances are set forth in the accompanying drawings, the claims, and the description herein. Other features, objects, and advantages of the inventive instances disclosed and contemplated herein can be combined with any other instance unless explicitly excluded.

In some of many aspects, the present disclosure provides methods of using genetic markers associated with endometriosis, for example via a computer-implemented program to predict risk of developing endometriosis, and methods of preventing or treating endometriosis or a symptom thereof. The methods disclosed herein can prevent or cancel an invasive procedure, such as a laparoscopy, that may otherwise have been performed on a subject but for the results, for example a (negative) diagnosis/prognosis, from the methods disclosed herein performed on the subject.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

In some cases, the present disclosure provides a method of testing for endometriosis and of treating a patient having at least one genetic mutation in at least one gene of UGT2B28, USP17L2 (alias DUBS), and METTL11B such that the patient is prevented from developing endometriosis or such that endometriosis in the patient is prevented from progressing. The treatment may be a surgical procedure, a hormone treatment, a pharmaceutical treatment, or a combination thereof. Further, the surgical procedure may be for instance a laparoscopy or the surgical removal of an endometriotic lesion and the pharmaceutical treatment may be for instance the administration of an oral contraceptive.

In some cases, genetic markers disclosed herein can be used for early diagnosis and prognosis of endometriosis, as well as early clinical intervention to mitigate progression of the disease. The use of these genetic markers can allow selection of subjects for clinical trials involving novel treatment methods. In some instances, genetic markers disclosed herein can be used to predict endometriosis and endometriosis progression, for example in treatment decisions for individuals who are recognized as having endometriosis. In some instances, genetic markers disclosed herein can enable prognosis of endometriosis in much larger populations compared with the populations which can currently be evaluated by using existing risk factors and biomarkers.

In some cases, disclosed herein is a method for endometriosis diagnosis/prognosis that can utilize detection of endometriosis associated biomarkers such as single nucleotide polymorphisms (SNPs), insertion deletion polymorphisms (indels), damaging mutation variants, loss of function variants, synonymous mutation variants, nonsynonymous mutation variants, nonsense mutations, recessive markers, splicing/splice-site variants, frameshift mutations, insertions, deletions, genomic rearrangements, stop-gain, stop-loss, Rare Variants (RVs), some of which are identified in Tables 1-2 (or diagnostically and predicatively functionally comparable biomarkers). In some instances, the method can comprise using a statistical assessment method such as Multi Dimensional Scaling analysis (MDS), logistic regression, or Bayesian analysis.

In some cases, disclosed herein is a treatment method to a subject determined to have or be predisposed to endometriosis. In some instances, the method can comprise administering to the subject a hormone therapy or an assisted reproductive therapy. In some instances, the method can comprise administering to the subject a therapy that at least partially compensates for endometriosis, prevents or reduces the severity of endometriosis that the subject may otherwise develop, or prevents endometriosis related complications, cancers, or associated disorders.

In some cases, provided herein is identification of new variants such as SNPs or indels, unique combinations of such variants, and haplotypes of variants that are associated with endometriosis and related pathologies. In some instances, the polymorphisms disclosed herein can be directly useful as targets for the design of diagnostic reagents and the development of therapeutic agents for use in the diagnosis and treatment of endometriosis and related pathologies. Based on the identification of variants associated with endometriosis, the present disclosure can provide methods of detecting these variants as well as the design and preparation of detection reagents needed to accomplish this task. Provided herein are novel variants in genetic sequences involved in endometriosis, methods of detecting these variants in a test sample, methods of identifying individuals who have an altered risk of developing endometriosis and for suggesting treatment options for endometriosis based on the presence of a variant(s) disclosed herein or its encoded product and methods of identifying individuals who are more or less likely to respond to a treatment.

In some cases, provided herein are variants such as SNPs and indels associated with endometriosis, nucleic acid molecules containing variants, methods and reagents for the detection of the variants disclosed herein, uses of these variants for the development of detection reagents, and assays or kits that utilize such reagents. In some instances, the variants disclosed herein can be useful for diagnosing, screening for, and evaluating predisposition to endometriosis and progression of endometriosis. In some instances, the variants can be useful in the determining individual subject treatment plans and design of clinical trials of devices for possible use in the treatment of endometriosis. In some instances, the variants and their encoded products can be useful targets for the development of therapeutic agents. In some instances, the variants combined with other non-genetic clinical factors can be useful for diagnosing, screening, evaluating predisposition to endometriosis, assessing risk of progression of endometriosis, determining individual subject treatment plans and design of clinical trials of devices for possible use in the treatment of endometriosis. In some instances, the variants can be useful in the selection of recipients for an oral contraceptive type therapeutic.

### Definitions

Unless otherwise indicated, open terms for example “contain,” “containing,” “include,” “including,” and the like mean comprising.

The singular forms “a”, “an”, and “the” are used herein to include plural references unless the context clearly dictates otherwise. Accordingly, unless the contrary is indicated, the numerical parameters set forth in this application are approximations that may vary depending upon the desired properties sought to be obtained by the present invention.

Unless otherwise indicated, some instances herein contemplate numerical ranges. When a numerical range is provided, unless otherwise indicated, the range includes the range endpoints. Unless otherwise indicated, numerical ranges include all values and subranges therein as if explicitly written out. Unless otherwise indicated, any numerical ranges and/or values herein, following or not following the term “about,” can be at 85-115% (i.e., plus or minus 15%) of the numerical ranges and/or values.

As used herein, “endometriosis” refers to any nonmalignant disorder in which functioning endometrial tissue is present in a location in the body other than the endometrium of the uterus, i.e. outside the uterine cavity or is present within the myometrium of the uterus. For purposes herein it also includes conditions, such as adenomyosis/adenomyoma, that exhibit myometrial tissue in the lesions. Endometriosis can include endometriosis externa, endometrioma, adenomyosis, adenomyomas, adenomyotic nodules of the uterosacral ligaments, endometriotic nodules other than of the uterosacral ligaments, autoimmune endometriosis, mild endometriosis, moderate endometriosis, severe endometriosis, superficial (peritoneal) endometriosis, deep (invasive) endometriosis, ovarian endometriosis, endometriosis-related cancers, and/or “endometriosis-associated conditions”. Unless stated otherwise, the term endometriosis is used herein to describe any of these conditions.

As used herein, “treatment” includes one or more of: reducing the frequency and/or severity of symptoms, elimination of symptoms and/or their underlying cause, and improvement or remediation of damage. For example, treatment of endometriosis includes, for example, relieving the pain experienced by a woman suffering from endometriosis, and/or causing the regression or disappearance of endometriotic lesions.

As used herein, a “therapeutic” can include a medical device, a pharmaceutical composition, a medical procedure, or any combination thereof. In some embodiments, a medical device may comprise a spinal brace. In some embodiments a medical device may comprise an artificial disc device. A medical device may comprise a surgical implant. A pharmaceutical composition may comprise a muscle relaxant, an anti-depressant, a steroid, an opioid, a cannabis-based therapeutic, acetaminophen, a non-steroidal anti-inflammatory, a neuropathic agent, a cannabis, a progestin, a progesterone, or any combination thereof. A neuropathic agent may comprise gabapentin. A non-steroidal anti-inflammatory may comprise naproxen, ibuprofen, a COX-2 inhibitor, or any combination thereof. A pharmaceutical composition may comprises a biologic agent, cellular therapy, regenerative medicine therapy, a tissue engineering approach, a stem cell transplantation or any combination thereof. A medical procedure may comprise an epidural injection (such as a steroid injection), acupuncture, exercise, physical therapy, an ultrasound, a radiofrequency ablation, a surgical therapy, a chiropractic manipulation, an osteopathic manipulation, or any combination thereof. A therapeutic can include a regenerative therapy such as a protein, a stem cell, a cord blood cell, an umbilical cord tissue, a tissue, or any combination thereof. A therapeutic can include cannabis. A therapeutic can include a biosimilar.

“Haplotype” can mean a combination of genotypes on the same chromosome or different chromosome occurring in a linkage disequilibrium block. Haplotypes serve as markers for linkage disequilibrium blocks, and at the same time provide information about the arrangement of genotypes within the blocks. Typing of only certain variants which serve as tags can, therefore, reveal all genotypes for variants located within a block. Thus, the use of haplotypes greatly facilitates identification of candidate genes associated with diseases and drug sensitivity.

“Linkage disequilibrium” or “LD” can mean that a particular combination of alleles (alternative nucleotides) or genetic variants for example at two or more different SNP (or RV) sites are non-randomly co-inherited (i.e., the combination of alleles at the different SNP (or RV) sites occurs more or less frequently in a population than the separate frequencies of occurrence of each allele or the frequency of a random formation of haplotypes from alleles in a given population). The term “LD” can differ from “linkage,” which describes the association of two or more loci on a chromosome with limited recombination between them. LD can also be used to refer to any non-random genetic association between allele(s) at two or more different SNP (or RV) sites. In some instances, when a genetic marker (e.g. SNP or RV) is identified as the genetic marker associated with a disease (in this instance endometriosis), it can be the minor allele (MA) of the particular genetic marker that is associated with the disease. In some instances, if the Odds Ratio (OR) of the MA is greater than 1.0, the MA of the genetic marker (in this instance the endometriosis associated genetic marker) can be correlated with an increased risk of endometriosis in a case subject as compared to a control subject and can be considered a causative marker (C), and if the OR of the MA less than 1.0, the MA of the genetic marker can be correlated with a decreased risk of endometriosis in a case subject as compared to a control subject and can be considered a protective marker (P). “Linkage disequilibrium block” or “LD block” can mean a region of the genome that contains multiple variants located in proximity to each other and that are transmitted as a block.

As used herein, “linkage disequilibrium” or “LD” may include a particular combination of alleles (alternative nucleotides) or genetic markers at two or more different SNP sites may be non-randomly co-inherited (i.e., the combination of alleles at the different SNP sites occurs more or less frequently in a population than the separate frequencies of occurrence of each allele or the frequency of a random formation of haplotypes from alleles in a given population). The term “LD” may differ from “linkage,” which describes the association of two or more loci on a chromosome with limited recombination between them. LD may also be used to refer to any non-random genetic association between allele(s) at two or more different SNP sites. Therefore, when a SNP may be in LD with other SNPs, the particular allele of the first SNP often predicts which SNP sites may be present in those alleles in LD. LD may be generally, but not exclusively, due to the physical proximity of the two loci along a chromosome. Hence, genotyping one of the SNP sites may give almost the same information as genotyping the other SNP site that may be in LD. Linkage disequilibrium may be caused by fitness interactions between genes or by such non-adaptive processes as population structure, inbreeding, and stochastic effects.

Various degrees of LD can be encountered between two or more SNPs with the result being that some SNPs may be more closely associated (i.e., in stronger LD) than others. Furthermore, the physical distance over which LD extends along a chromosome differs between different regions of the genome, and therefore the degree of physical separation 20 between two or more SNP sites necessary for LD to occur can differ between different regions of the genome. In one definition, LD can be described mathematically as SNPs that have a D prime value=1 and a LOD score>2.0 or an r-squared value>0.8.

As used herein, “linkage disequilibrium block” may include a region of the genome that contains multiple SNPs located in proximity to each other and that may be transmitted as a block.

As used herein, “D prime” or D′ (also referred to as the “linkage disequilibrium measure” or “linkage disequilibrium parameter”) may include the deviation of the observed allele frequencies from the expected, and may be a statistical measure of how well a biometric system can discriminate between different individuals. The larger the D′ value, the better a biometric system may be at discriminating between individuals.

As used herein, “LOD score” may include the “logarithm of the odd” score, which may be a statistical estimate of whether two genetic loci may be physically near enough to each other (or “linked”) on a particular chromosome that they may be likely to be inherited together. A LOD score of three or more may be generally considered statistically significant evidence of linkage.

As used herein, “R-squared” or “r2” (also referred to as “correlation coefficient”) may include a statistical measure of the degree to which two markers may be related. The nearer to 1.0 the r2 value is, the more closely the markers may be related to each other. R2 cannot exceed 1.0. D prime and LOD scores generally follow the above definition for SNPs in LD. R2, however, displays a more complex pattern and can vary between about 0.0003 and 1.0 in SNPs that may be in LD. (International HapMap Consortium, Nature Oct. 27, 2005; 437:1299-1320).

Biological samples obtained from individuals (e.g., human subjects) may be any sample from which a genetic material (e.g., nucleic acid sample) may be derived. Samples/Genetic materials may be from biopsy, fine needle aspirate sample, gynecological tissue, endometrial tissue, ovarian tissue, uterine tissue, cervical tissue, buccal swabs, saliva, blood, hair, nail, skin, cell, or any other type of tissue sample. In some instances, the genetic material (e.g., nucleic acid sample) comprises mRNA, cDNA, genomic DNA, or PCR amplified products produced therefrom, or any combination thereof. In some instances, the genetic material (e.g., nucleic acid sample) comprises PCR amplified nucleic acids produced from cDNA or mRNA. In some instances, the genetic material (e.g., nucleic acid sample) comprises PCR amplified nucleic acids produced from genomic DNA. In some embodiments, the genetic material comprises a protein sample. In some embodiments, the sample may comprise a cell-free sample.

As used herein, the term “cell-free” or “cell free” may refer to the condition of the nucleic acid sequence as it appeared in the body before the sample may be obtained from the body. For example, circulating cell-free nucleic acid sequences in a sample may have originated as cell-free nucleic acid sequences circulating in the bloodstream of the human body. In contrast, nucleic acid sequences that may be extracted from a solid tissue, such as a biopsy, may be generally not considered to be “cell-free.” In some embodiments, cell-free DNA may comprise fetal DNA, maternal DNA, or a combination thereof. In some embodiments, cell-free DNA may comprise DNA fragments released into a blood plasma. In some embodiments, cell-free DNA may comprise circulating tumor DNA. In some embodiments, cell-free DNA may comprise circulating DNA indicative of a tissue origin, a disease or a condition. A cell-free nucleic acid sequence may be isolated from a blood sample. A cell-free nucleic acid sequence may be isolated from a plasma sample. A cell-free nucleic acid sequence may comprise a complementary DNA (cDNA). In some embodiments, one or more cDNAs may form a cDNA library.

The term “subject,” as used herein, may be any animal or living organism. Animals can be mammals, such as humans, non-human primates, rodents such as mice and rats, dogs, cats, pigs, sheep, rabbits, and others. A subject may be a dog. A subject may be a human. Animals can be fish, reptiles, or others, Animals can be neonatal, infant, adolescent, or adult animals. Humans can be more than about: 1, 2, 5, 10, 20, 30, 40, 50, 60, 65, 70, 75, or about 80 years of age. The subject may have or be suspected of having a condition or a disease, such as endometriosis or related condition. The subject may be a patient, such as a patient being treated for a condition or a disease, such as a patient suffering from endometriosis. The subject may be predisposed to a risk of developing a condition or a disease such as endometriosis. The subject may be in remission from a condition or a disease, such as a patient recovering from endometriosis. The subject may be healthy. The subject may be a subject in need thereof. The subject may be a female subject or a male subject.

The term “sequencing” as used herein, may comprise high-throughput sequencing, next-gen sequencing, Maxam-Gilbert sequencing, massively parallel signature sequencing, Polony sequencing, 454 pyrosequencing, pH sequencing, Sanger sequencing (chain termination), Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, Enigma sequencing, sequencing-by-hybridization, sequencing-by-ligation, or any combination thereof. The sequencing output data may be subject to quality controls, including filtering for quality (e.g., confidence) of base reads. Exemplary sequencing systems include 454 pyrosequencing (454 Life Sciences), Illumina (Solexa) sequencing, SOLiD (Applied Biosystems), and Ion Torrent Systems' pH sequencing system. In some cases, a nucleic acid of a sample may be sequenced without an associated label or tag. In some cases, a nucleic acid of a sample may be sequenced, the nucleic acid of which may have a label or tag associated with it.

Nanopores may be used to sequence, a sample, a small portion (such as one full gene or a portion of one gene), a substantial portion (such as multiple genes or multiple chromosomes), or the entire genomic sequence of an individual. Nanopore sequencing technology may be commercially available or under development from Sequenom (San Diego, Calif.), Illumina (San Diego, Calif.), Oxford Nanopore Technologies LTD (Kidlington, United Kingdom), and Agilent Laboratories (Santa Clara, Calif.). Nanopore sequencing methods and apparatus may be described in the art and may be provided in U.S. Pat. No. 5,795,782, herein incorporated by reference in its entirety.

Nanopore sequencing can use electrophoresis to transport a sample through a pore. A nanopore system may contain an electrolytic solution such that when a constant electric field is applied, an electric current can be observed in the system. The magnitude of the electric current density across a nanopore surface may depend on the nanopore's dimensions and the composition of the sample that is occupying the nanopore. During nanopore sequencing, when a sample approaches and or goes through the nanopore, the samples may cause characteristic changes in electric current density across nanopore surfaces, these characteristic changes in the electric current enables identification of the sample. Nanopores used herein may be solid-state nanopores, protein nanopores, or hybrid nanopores comprising protein nanopores or organic nanotubes such as carbon or graphene nanotubes, configured in a solid-state membrane, or like framework. In some embodiments, nanopore sequencing can be biological, a solid state nanopore or a hybrid biological/solid state nanopore.

In some instances, a biological nanopore can comprise transmembrane proteins that may be embedded in lipid membranes. In some embodiments, a nanopore described herein may comprise alpha hemolysin. In some embodiments, a nanopore described herein may comprise Mycobacterium smegmatis porin.

Solid state nanopores do not incorporate proteins into their systems. Instead, solid state nanopore technology uses various metal or metal alloy substrates with nanometer sized pores that allow samples to pass through. Solid state nanopores may be fabricated in a variety of materials including but not limited to, silicon nitride (Si₃N₄), silicon dioxide (SiO₂), and the like. In some instances, nanopore sequencing may comprise use of tunneling current, wherein a measurement of electron tunneling through bases as sample (ssDNA) translocates through the nanopore is obtained. In some embodiments, a nanopore system can have solid state pores with single walled carbon nanotubes across the diameter of the pore. In some embodiments, nanoelectrodes may be used on a nanopore system described herein. In some embodiments, fluorescence can be used with nanopores, for example solid state nanopores and fluorescence. In such a system the fluorescence sequencing method converts each base of a sample into a characteristic representation of multiple nucleotides which bind to a fluorescent probe strand-forming dsDNA (were the sample comprises DNA). Where a two color system is used, each base can be identified by two separate fluorescences, and will therefore be converted into two specific sequences. Probes may consist of a fluorophore and quencher at the start and end of each sequence, respectively. Each fluorophore may be extinguished by the quencher at the end of the preceding sequence. When the dsDNA is translocating through a solid state nanopore, the probe strand may be stripped off, and the upstream fluorophore will fluoresce.

In some embodiments, a nanopore can comprise from about 1 nm to about 100 nm channel or an aperture may be formed through a solid substrate, usually a planar substrate, such as a membrane, through which an analyte, such as single stranded DNA, may be induced to translocate. In other embodiments, a nanopore can comprise from about 2 nm to about 50 nm channel or aperture formed through a substrate; and in still other embodiments, from about 2 nm to about 30 nm, or from about 2 nm to about 20 nm, or from about 3 nm to about 30 nm, or from about 3 nm to about 20 nm, or from about 3 nm to about 10 nm channel or aperture is formed through a substrate.

In some embodiments, nanopores used in connection with the methods and devices of the disclosure may be provided in the form of arrays, such as an array of clusters of nanopores, which may be disposed regularly on a planar surface. In some embodiments, clusters may each be in a separate resolution limited area so that optical signals from nanopores of different clusters are distinguishable by the optical detection system employed, but optical signals from nanopores within the same cluster cannot necessarily be assigned to a specific nanopore within such cluster by the optical detection system employed.

In some instances, the gene sequence may be mapped with one or more reference sequences to identify sequence variants. The base reads may be mapped against a reference sequence, which in various embodiments may be presumed to be a “normal” non-disease sequence. The DNS sequence derived from the Human Genome Project is generally used as a “premier” reference sequence. A number of mapping applications are known, and include TMAP, BWA, GSMAPPER, ELAND, MOSAIK, and MAQ. Various other alignment tools are known, and may also be implemented to map the base reads.

In some cases, based on the sequence alignments, and mapping results, sequence variants can be identified. Types of variants may include insertions, deletions, indels (a colocalized insertion and deletion), damaging mutation variants, loss of function variants, synonymous mutation variants, nonsynonymous mutation variants, nonsense mutations, recessive markers, splicing/splice-site variants, frameshift mutation, insertions, deletions, genomic rearrangements, stop-gain, stop-loss, Rare Variants (RVs), translocations, inversions, and substitutions. While the type of variants analyzed is not limited, the most numerous of the variant types will be single nucleotide substitutions, for which a wealth of data is currently available. In various embodiments, comparison of the test sequence with the reference sequence will produce at least 500 variants, at least 1000 variants, at least 3,000 variants, at least 5,000 variants, at least 10,000 variants, at least 20,000 variants, or at least 50,000 variants, but in some embodiments, will produce at least 1 million variants, at least 2 million variants, at least 3 million variants, at least 4 million variants, or at least 10 million variants. The tools provided herein enable the user to navigate the vast amounts of genetic data to identify potentially disease-causing variants.

In some cases, a wealth of data can be extracted for the identified variants, including one or more of conservation scores, genic/genomic location, zygosity, SNP ID, Polyphen, FATHMM, LRT, Mutation Accessor, and SIFT predictions, splice site predictions, amino acid properties, disease associations, annotations for known variants, variant or allele frequency data, and gene annotations. Data may be calculated and/or extracted from one or more internal or external databases. Since certain categories of annotations (e.g., amino acid properties/PolyPhen and SIFT data) are dependent on a nature of the region of the genome in which they are contained (e.g., whether a variant is contained within a region translated to give rise to an amino acid sequence in a resultant protein), these annotations can be carried out for each known transcript. Exemplary external databases include OMIM (Online Mendelian Inheritance in Man), HGMD (The Human Gene Mutation Databse), PubMed, PolyPhen, SIFT, SpliceSite, reference genome databases, the University of California Santa Cruz (UCSC) genome database, CLINVAR database, the BioBase biological databases, the dbSNP Short Genetic Variations database, the Rat Genome Database (RGD), and/or the like. Various other databases may be employed for extracting data on identified variants. Variant information may be further stored in a central data repository, and the data extracted for future sequence analyses.

The term “homology” can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which may correspond with a particular nucleic acid sequence described herein), such particular polypeptide sequence can be determined using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity is calculated over the full length of the reference sequence and that gaps in homology of up to 5% of the total reference sequence are allowed.

In some embodiments, the identity between a reference sequence (query sequence, i.e., a sequence of the present disclosure) and a subject sequence, also referred to as a global sequence alignment, may be determined using the FASTDB computer program based on the algorithm of Brutlag et al. (Comp. App. Biosci. 6:237-245 (1990)). In some embodiments, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some embodiments, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. A 90 residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

Analysis of Rare and Private Mutations in Sequenced Endometriosis Genes

In some cases, the present disclosure provides an analysis to evaluate a coding region of a gene as a component of a genetic diagnostic or predictive test for endometriosis. In some instances, the analysis can comprise one or more of the approaches disclosed herein.

In some instances, the analysis can comprise performing DNA variant search on the next generation sequencing output file using a standard software designed for this purpose, for example Life Technologies/Thermo Fisher TMAP algorithm with their default parameter settings, and Life Technologies/Thermo Fisher Torrent Variant Caller software. ANNOVAR can be used to classify coding variants as synonymous, missense, frameshift, splicing, stop-gain, or stop-loss. Variants can be considered “loss-of-function” if the variant causes a stop-loss, stop-gain, splicing, or frame-shift insertion or deletion).

In some instances, the analysis can comprise evaluating prediction of an effect of each variant on protein function in silico using a variety of different software algorithms: Polyphen 2, Sift, Mutation Accessor, Mutation Taster, FATHMM, LRT, MetaLR, or any combination thereof. Missense variants can be deemed “damaging” if they are predicted to be damaging by at least one of the seven algorithms tested.

In some instances, the analysis can comprise searching population databases (e.g., gnomAD) and proprietary endometriosis allele frequency databases for the prevalence of any loss of function or damaging mutations identified by these analyses. The log of the odds ratio can be used to weight the marker when the variant has been previously observed in the reference databases. When a damaging variant or loss of function variant has not been reported in the reference databases, a default odds ratio of 10 can be used to weight the finding.

In some instances, the analysis can comprise incorporating findings into the Risk Score as with the other low-frequency alleles. Risk Score=Summation [log(OR)×Count], where count equals the number of low frequency alleles detected at each endometriosis associated locus. Risk scores can be converted to probability using a nomogram based on confirmed diagnoses.

In some instances, the methods of the present disclosure can provide a high sensitivity of detecting gene mutations and diagnosing endometriosis that is greater than 60%, 65%, 70%, 75%, 80%, 85%, 90%, 91%, 92%, 93%, 94%, 95%, 95.5%, 96%, 96.5%, 97%, 97.5%, 98%, 98.5%, 99%, 99.5% or more. In some instances, the methods disclosed herein can provide a high specificity of detecting and classifying gene mutations and endometriosis, for example, greater than 80%, 85%, 90%, 91%, 92%, 93%, 94%, 95%, 95.5%, 96%, 96.5%, 97%, 97.5%, 98%, 98.5%, 99%, 99.5% or more. In some instances, a nominal specificity for the method disclosed herein can be greater than or equal to 70%. In some instances, a nominal Negative Predictive Value (NPV) for the method disclosed herein can be greater than or equal to 95%. In some instances, a NPV for the method disclosed herein can be about 95%, 95.5%, 96%, 96.5%, 97%, 97.5%, 98%, 98.5%, 99%, 99.5% or more. In some instances, a nominal Positive Predictive Value (PPV) for the method disclosed herein can be greater than or equal to 95%. In some instances, a PPV for the method disclosed herein can be about 95%, 95.5%, 96%, 96.5%, 97%, 97.5%, 98%, 98.5%, 99%, 99.5% or more. In some instances, the accuracy of the methods disclosed herein in diagnosing endometriosis can be greater than 70%, 75%, 80%, 85%, 90%, 91%, 92%, 93%, 94%, 95%, 95.5%, 96%, 96.5%, 97%, 97.5%, 98%, 98.5%, 99%, 99.5% or more.

Computer Implemented Methods

In some aspects, the present disclosure provides methods for analysis of gene sequence data associated software and computer systems (e.g., cloud-based). The method, for example being computer implemented, can enable a clinical geneticist or other healthcare technician to sift through vast amounts of gene sequence data, to identify potential disease-causing genomic variants. In some cases, the gene sequence data is from a patient who may be suspected of having a genetic disorder such as endometriosis.

In some cases, provided herein is a method for identifying a genetic disorder such as endometriosis or predicting a risk thereof in an individual, or identifying a genetic variant that is causative of a phenotype in an individual. In some instances, the method can comprise determining gene sequence for a patient suspected of having a genetic disorder, identifying sequence variants, annotating the identified variants based on one or more criteria, and filtering or searching the variants at least partially based on the annotations, to thereby identify potential disease-causing variants.

In some instances, the gene sequence is obtained by use of a sequencing instrument, or alternatively, gene sequence data is obtained from another source, such as for example, a commercial sequencing service provider. Gene sequence can be chromosomal sequence, cDNA sequence, or any nucleotide sequence information that allows for detection of genetic disease. Generally, the amount of sequence information is such that computational tools may be required for data analysis. For example, the sequence data may represent at least half of the individual's genomic or cDNA sequence (e.g., of a representative cell population or tissue), or the individuals entire genomic or cDNA sequence. In various embodiments, the sequence data comprises the nucleotide sequence for at least 1 million base pairs, at least 10 million base pairs, or at least 50 million base pairs. In certain embodiments, the DNA sequence is the individual's exome sequence or full exonic sequence component (i.e., the exome; sequence for each of the exons in each of the known genes in the entire genome). In some embodiments, the source of genomic DNA or cDNA may be any suitable source, and may be a sample particularly indicative of a disease or phenotype of interest, including blood cells (e.g, PBMCs, or a T-cell or B-cell population). In certain embodiments, the source of the sample is a tissue or sample that is potentially malignant.

In some instances, whole genome sequence can comprise the entire sequence (including all chromosomes) of an individual's germline genome. In some embodiments, the concatenated length for a whole genome sequence is approximately 3.2 Gbases or 3.2 billion nucleotides.

In some instances, the gene sequence may be determined by any suitable method. For example, the gene sequence may be a cDNA sequence determined by clonal amplification (e.g., emulsion PCR) and sequencing. Base calling may be conducted based on any available method, including Sanger sequencing (chain termination), pH sequencing, pyrosequencing, sequencing-by-hybridization, sequencing-by-ligation, etc. The sequencing output data may be subject to quality controls, including filtering for quality (e.g., confidence) of base reads. Exemplary sequencing systems include 454 pyrosequencing (454 Life Sciences), Illumina (Solexa) sequencing, SOLiD (Applied Biosystems), and Ion Torrent Systems' pH sequencing system.

In some instances, the gene sequence may be mapped with one or more reference sequences to identify sequence variants. For example, the base reads are mapped against a reference sequence, which in various embodiments is presumed to be a “normal” non-disease sequence. The DNS sequence derived from the Human Genome Project is generally used as a “premier” reference sequence. A number of mapping applications are known, and include TMAP, BWA, GSMAPPER, ELAND, MOSAIK, and MAQ. Various other alignment tools are known, and may also be implemented to map the base reads.

In some cases, based on the sequence alignments, and mapping results, sequence variants can be identified. Types of variants may include insertions, deletions, indels (a colocalized insertion and deletion), damaging mutation variants, loss of function variants, synonymous mutation variants, nonsynonymous mutation variants, nonsense mutations, recessive markers, splicing/splice-site variants, frameshift mutation, insertions, deletions, genomic rearrangements, stop-gain, stop-loss, Rare Variants (RVs), translocations, inversions, and substitutions. While the type of variants analyzed is not limited, the most numerous of the variant types will be single nucleotide substitutions, for which a wealth of data is currently available. In various embodiments, comparison of the test sequence with the reference sequence will produce at least 500 variants, at least 1000 variants, at least 3,000 variants, at least 5,000 variants, at least 10,000 variants, at least 20,000 variants, or at least 50,000 variants, but in some embodiments, will produce at least 1 million variants, at least 2 million variants, at least 3 million variants, at least 4 million variants, or at least 10 million variants. The tools provided herein enable the user to navigate the vast amounts of genetic data to identify potentially disease-causing variants.

In some cases, a wealth of data can be extracted for the identified variants, including one or more of conservation scores, genic/genomic location, zygosity, SNP ID, Polyphen, FATHMM, LRT, Mutation Accessor, and SIFT predictions, splice site predictions, amino acid properties, disease associations, annotations for known variants, variant or allele frequency data, and gene annotations. Data may be calculated and/or extracted from one or more internal or external databases. Since certain categories of annotations (e.g., amino acid properties/PolyPhen and SIFT data) are dependent on a nature of the region of the genome in which they are contained (e.g., whether a variant is contained within a region translated to give rise to an amino acid sequence in a resultant protein), these annotations can be carried out for each known transcript. Exemplary external databases include OMIM (Online Mendelian Inheritance in Man), HGMD (The Human Gene Mutation Databse), PubMed, PolyPhen, SIFT, SpliceSite, reference genome databases, the University of California Santa Cruz (UCSC) genome database, CLINVAR database, the BioBase biological databases, the dbSNP Short Genetic Variations database, the Rat Genome Database (RGD), and/or the like. Various other databases may be employed for extracting data on identified variants. Variant information may be further stored in a central data repository, and the data extracted for future sequence analyses.

In some instances, variants may be tagged by the user with additional descriptive information to aid subsequent analysis. For example, confidence in the existence of the variant can be recorded as confirmed, preliminary, or sequence artifact. Certain sequencing technologies have a tendency to produce certain types of sequence artifacts, and the method herein can allow such suspected artifacts to be recorded. The variants may be further tagged in basic categories of benign, pathogenic, or unknown, or as potentially of interest.

In some instances, queries can be run to identify variants meeting certain criteria, or variant report pages can be browsed by chromosomal position or by gene, the latter allowing researchers to focus on only those variations that exist in a particular set of genes of interest. In some embodiments, the user selects only variants with well-documented and published disease associations (e.g., by filtering based on HGMD or other disease annotation). Alternatively, the user can filter for variants not previously associated with disease, but of a type likely to be deleterious, such as those introducing frameshifts, non-synonymous substitutions (predicted by Polyphen or SIFT), or premature terminations. Further, the user can exclude from analysis those variants believed to be neutral (based on their frequency of occurrence in studies populations), for example, through exclusion of variants in dbSNP. Additional exclusion criteria include mode of inheritance (e.g., heterozygosity), depth of coverage, and quality score.

In certain embodiments, base calling is carried out to extract the sequence of the sequencing reads from an image file produced by an instrument scanner. Following base calling and base quality trimming/filtering, the reads are mapped against a reference sequence (assumed to be normal for the phenotype under analysis) to identify variations (variants) between the two with the assumption that one or more of these differences will be associated with phenotype of the individual whose DNA is under analysis. Subsequently, each variant is annotated with data that can be used to determine the likelihood that that particular variant is associated with the phenotype under analysis. The analysis may be fully or partially automated as described in detail below, and may include use of a central repository for data storage and analysis, and to present the data to analysts and clinical geneticists in a format that makes identification of variants with a high likelihood of being associated with the phenotypic difference more efficient and effective.

In some embodiments, a user can be provided with the ability to run cross sample queries where the variants from multiple samples are interrogated simultaneously. In such embodiments, for example, a user can build a query to return data on only those variants that are exactly shared across a user defined group of samples. This can be useful for family based analyses where the same variant is believed to be associated with disease in each of the affected family members. For another example, the user can also build a query to return only those variants that are present in genes where the gene contains at least one, but not necessarily the same, variant. This can be useful where a group of individuals with disease are not related (the variants associated with the disease are not necessary exactly the same, but result in an alteration in normal function). For yet another example, the user can specify to ignore genes containing variants in a user defined group of samples. This can be useful to exclude polymorphisms (variants believed or confirmed not to be associated with disease) where the user has access to a user defined group of control individuals who are believed to not have the disease associated variant. For each of these queries a user can additionally filter the variants by specifying any or all of the previously discussed filters on top of the cross sample analyses. This allows a user to identify variants matching these criteria, which are shared between or segregated amongst samples.

For example, a variant analysis system can be implemented locally, or implemented using a host device and a network or cloud computing. For example, the variant analysis system can be software stored in memory of a personal computing device (PC) and implemented by a processor of the PC. In such embodiments, for example, the PC can download the software from a host device and/or install the software using any suitable device such as a compact disc (CD).

The method may employ a computer-readable medium, or non-transitory processor-readable medium. Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

Examples of computer code can include, but are not limited to, micro-code or micro-instructions, machine instructions, such as produced by a compiler, code used to produce a web service, and files containing higher-level instructions that are executed by a computer using an interpreter. For example, embodiments may be implemented using Python, Java, C++, or other programming languages (e.g., object-oriented programming languages) and development tools. Additional examples of computer code can include, but are not limited to, control signals, encrypted code, and compressed code.

In some cases, variants provided herein may be “provided” in a variety of mediums to facilitate use thereof. As used in this section, “provided” refers to a manufacture, other than an isolated nucleic acid molecule, that contains variant information of the present disclosure. Such a manufacture provides the variant information in a form that allows a skilled artisan to examine the manufacture using means not directly applicable to examining the variants or a subset thereof as they exist in nature or in purified form. The variant information that may be provided in such a form includes any of the variant information provided by the present disclosure such as, for example, polymorphic nucleic acid and/or amino acid sequence information, information about observed variant alleles, alternative codons, populations, allele frequencies, variant types, and/or affected proteins, or any other information provided herein.

In some instances, the variants can be recorded on a computer readable medium. As used herein, “computer readable medium” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon a nucleotide sequence of the present disclosure. One such medium is provided with the present application, namely, the present application contains computer readable medium (CD-R) that has nucleic acid sequences (and encoded protein sequences) containing variants provided/recorded thereon in ASCII text format in a Sequence Listing along with accompanying tables that contain detailed variant and sequence information.

As used herein, “recorded” can refer to a process for storing information on computer readable medium. A skilled artisan can readily adopt any of the presently known methods for recording information on computer readable medium to generate manufactures comprising the variant information of the present disclosure. A variety of data storage structures are available to a skilled artisan for creating a computer readable medium having recorded thereon a nucleotide or amino acid sequence of the present disclosure. The choice of the data storage structure will generally be based on the means chosen to access the stored information. In addition, a variety of data processor programs and formats can be used to store the nucleotide/amino acid sequence information of the present disclosure on computer readable medium. For example, the sequence information can be represented in a word processing text file, formatted in commercially-available software such as WordPerfect and Microsoft Word, represented in the form of an ASCII file, or stored in a database application, such as OB2, Sybase, Oracle, or the like. A skilled artisan can readily adapt any number of data processor structuring formats (e.g., text file or database) in order to obtain computer readable medium having recorded thereon the variant information of the present disclosure.

By providing the variants in computer readable form, a skilled artisan can access the variant information for a variety of purposes. Computer software is publicly available which allows a skilled artisan to access sequence information provided in a computer readable medium. Examples of publicly available computer software include BLAST and BLAZE search algorithms.

In some cases, the present disclosure can provide systems, particularly computer-based systems, which contain the variant information described herein. Such systems may be designed to store and/or analyze information on, for example, a large number of variant positions, or information on variant genotypes from a large number of individuals. The variant information of the present disclosure represents a valuable information source. The variant information of the present disclosure stored/analyzed in a computer-based system (e.g., cloud-based) may be used for such computer-intensive applications as determining or analyzing variant allele frequencies in a population, mapping endometriosis genes, genotype-phenotype association studies, grouping variants into haplotypes, correlating variant haplotypes with response to particular treatments or for various other bioinformatic, pharmacogenomic or drug development.

As used herein, “a computer-based system” can refer to the hardware means, software means, and data storage means used to analyze the variant information of the present disclosure. The minimum hardware means of the computer-based systems of the present disclosure may comprise a central processing unit (CPU), input means, output means, and data storage means. A skilled artisan can readily appreciate that any one of the currently available computer-based systems are suitable for use in the present disclosure. Such a system can be changed into a system of the present disclosure by utilizing the variant information provided on the CD-R, or a subset thereof, without any experimentation.

As stated above, the computer-based systems can comprise a data storage means having stored therein variants of the present disclosure and the necessary hardware means and software means for supporting and implementing a search means. As used herein, “data storage means” refers to memory which can store variant information of the present disclosure, or a memory access means which can access manufactures having recorded thereon the variant information of the present disclosure.

As used herein, “search means” can refer to one or more programs or algorithms that are implemented on the computer-based system to identify or analyze variants in a target sequence based on the variant information stored within the data storage means. Search means can be used to determine which nucleotide is present at a particular variant position in the target sequence. As used herein, a “target sequence” can be any DNA sequence containing the variant position(s) to be searched or queried.

A variety of structural formats for the input and output means can be used to input and output the information in the computer-based systems of the present disclosure. An exemplary format for an output means is a display that depicts the presence or absence of specified nucleotides (alleles) at particular variant positions of interest. Such presentation can provide a rapid, binary scoring system for many variants simultaneously.

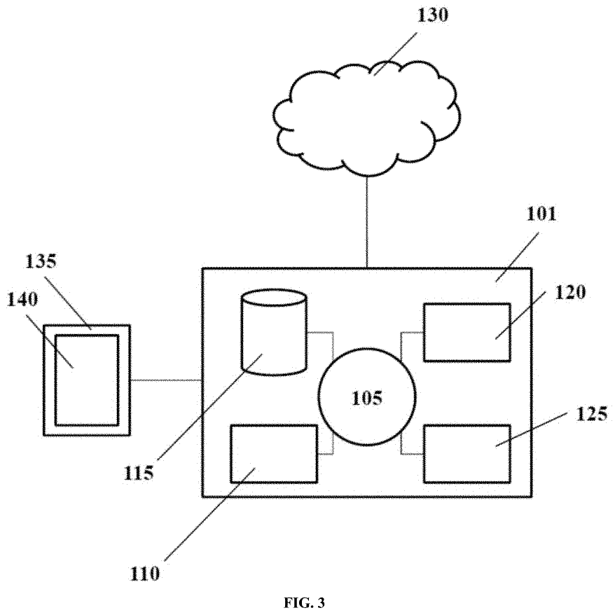

In some cases, the present disclosure provides computer-based systems that are programmed to implement methods of the disclosure. FIG. 3 shows a computer system 101 that can be programmed or configured for endometriosis diagnosis. The computer system 101 can regulate various aspects of detection of genetic variants associated with endometriosis of the present disclosure. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example a monitor. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105. The algorithm can, for example, Polyphen 2, Sift, Mutation Accessor, Mutation Taster, FATHMM, LRT, MetaLR, or any combination thereof.

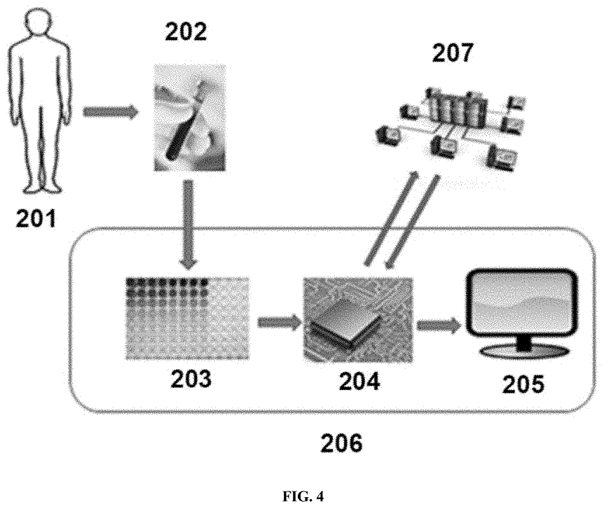

In some cases, as shown in FIG. 4, a sample 202 containing a genetic material may be obtained from a subject 201, such as a human subject. A sample 202 may be subjected to one or more methods as described herein, such as performing an assay. In some cases, an assay may comprise hybridization, amplification, sequencing, labeling, epigenetically modifying a base, or any combination thereof. One or more results from a method may be input into a processor 204. One or more input parameters such as a sample identification, subject identification, sample type, a reference, or other information may be input into a processor 204. One or more metrics from an assay may be input into a processor 204 such that the processor may produce a result, such as a diagnosis of endometriosis or a recommendation for a treatment. A processor may send a result, an input parameter, a metric, a reference, or any combination thereof to a display 205, such as a visual display or graphical user interface. A processor 204 may (i) send a result, an input parameter, a metric, or any combination thereof to a server 207, (ii) receive a result, an input parameter, a metric, or any combination thereof from a server 207, (iii) or a combination thereof.

Methods of Detection of Variants

The methods and kits as described herein may include detecting a presence of a variant allele. The variant allele detected may be a reference allele, an alternative allele, a non-reference allele, a major allele, a minor allele, or any combination thereof. In some cases, one or more minor alleles are detected. In some cases, a major allele is detected. In some cases, one or more minor alleles and a major allele are detected.

A major allele may be a variant allele that occurs with greater than 50% frequency in a population of subjects. A variant allele may or may not be a major allele depending on the population of subjects. A major allele may be present in about: 50.5%, 55%, 60%, 65%, 70%, 75%, 80%, 85%, 90%, 95%, 96%, 97%, 98%, 99%, 99.5% of a population. A major allele may be present in from about 50.5% to about 99.9% of a population. A major allele may be present in from about 50.5% to about 80% of a population. A major allele may be present in from about 50.5% to about 70% of a population. A major allele may be present in from about 50.5% to about 60% of a population. A major allele may be present in from about 55% to about 99.9% of a population. A major allele may be present in from about 60% to about 99.9% of a population. A major allele may be present in from about 70% to about 99.9% of a population. A major allele may be present in from about 80% to about 99.9% of a population.

A minor allele may be a variant allele that occurs with less than 50% frequency in a population of subjects. A variant allele may or may not be a minor allele depending on the population of subjects. A minor allele may be present in about: 49.5%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.01% of a population. A minor allele may be present in from about 49.5% to about 0.1% of a population. A minor allele may be present in from about 40% to about 0.1% of a population. A minor allele may be present in from about 30% to about 0.1% of a population. A minor allele may be present in from about 20% to about 0.1% of a population. A minor allele may be present in from about 10% to about 0.1% of a population. A minor allele may be present in from about 5% to about 0.1% of a population. A minor allele may be present in from about 1% to about 0.01% of a population. A minor allele may be present in from about 0.5% to about 0.01% of a population. A minor allele may be present in from about 0.3% to about 0.01% of a population. A minor allele may be present in from about 0.2% to about 0.01% of a population.

A reference allele may be selected or assigned. A reference allele may be a major allele. A reference allele may not be a major allele. A reference allele may be an ancestral allele. A reference allele may be a major allele from a general population of subjects. A reference allele may be compared to an alternative allele or non-reference allele. An alternative or non-reference allele may be a minor allele. An alternative or non-reference allele may not be a minor allele. In some cases, there may be more than one alternative or non-reference allele, such as 2, 3, 4, or more alternative or non-reference alleles. More than one alternative or non-reference allele may represent a plurality of minor alleles.

A reference allele, an alternative allele, a non-reference allele, a major allele, a minor allele, or any combination thereof may be defined by a population from which a variant allele is detected. A population of subjects may be representative of a general population. A population of subjects may be representative of individuals having been diagnosed with endometriosis or suffering from symptoms of endometriosis. A major and minor allele may vary depending on the population selected. A population may be defined by one or more of: a size, a distribution of: age, health status, gender, ethnicity, geographical location, or any combination thereof.

A population size may be about: 5, 10, 20, 30, 40, 50, 60, 70, 80, 90, 100, 125, 150, 175, 200, 250, 500, 1000, 2500, 5000, 10,000, 25,000, 50,000, 75,000, 100,000, 250,000, 500,000, 750,000, or 100,000,000 subjects. A population may comprise females, males, or both. A population may comprise healthy individuals or individuals having been diagnosed with a disease or condition or a combination thereof. A population may include individuals of a same ethnicity or a different ethnicity. A population may include individuals of a same geographical location or a different geographical location. A population may include infants, children, adolescents, young adults, middle aged adults, elderly subjects, or any combination thereof.

In some cases, a population may be representative of a general population or at least a portion of a general population. A population may be a global population. The reference allele may be the major allele, occurring in greater than 50% of the general population. The non-reference or alternative allele may be the minor allele, occurring in less than 50% of the general population, such as a rare minor allele occurring in less than about 5%, 4%, 3%, 2%, or 1% of a general population. Individuals identified as having the minor allele may be individuals that have an increased risk of developing endometriosis or individuals that have endometriosis.

In some cases, a population may be representative of a selected population of individuals, such as individuals suffering from endometriosis or having been previously diagnosed with endometriosis. The reference allele may be major allele, occurring in greater than 50% of the selected population. Individuals having the major allele may be indicative of a presence of endometriosis or a risk of developing endometriosis. The non-reference allele or alternative allele, occurring in less than 50% of the selected population may be indicative of non-diagnostic variant or indicative of a subtype of endometriosis that may occur in a subset of individuals.

In some aspects, the present disclosure provides methods to detect variants, e.g, detecting a genetic variant in a panel comprising two or more genetic variants defining a minor allele disclosed herein (e.g., in Table 1 or Table 2). In some instances, the detecting comprises, DNA sequencing, hybridization with a complementary probe, an oligonucleotide ligation assay, a PCR-based assay, or any combination thereof. In some instances, the panel comprises at least: 2, 3, 4, 5, 6, 7, 8, 9, 10, 15, 20, 25, 30, 35, 40, 45, 50, 60, 70, 75, 80, 90, 100, 150, 200, 250, 300, 350, 400, 450, 500, or more genetic variants defining minor alleles disclosed herein (e.g., in Table 1 or Table 2). In some instances, the genetic variant to detect or detected has an odds ratio (OR) of at least: 0.1, 1, 1.5, 2, 5, 10, 20, 50, 100, 127, 130, 140, 150, 200, 300, 400, 500, 600, 700, 800, 900, 1000, 1500, 2000, 2500, 3000, 3500, 4000, 4500, 5000, or more. In some embodiments, the OR is at least 127. In some instances, the panel to detect further comprises one or more protein damaging or loss of function variants in one or more genes selected from the group consisting of GAT2, CCDC169, CASP8AP2, POU2F3, CD19, IGSF3, GLI3, PEX26, OLIG3, CIB4, NKX3-2, CFTR, and any combinations thereof.

In some cases, variants of the present disclosure may include single nucleotide polymorphisms (SNPs), insertion deletion polymorphisms (indels), damaging mutation variants, loss of function variants, synonymous mutation variants, nonsynonymous mutation variants, nonsense mutations, recessive markers, splicing/splice-site variants, frameshift mutation, insertions, deletions, genomic rearrangements, stop-gain, stop-loss, Rare Variants (RVs), translocations, inversions, and substitutions.

Variants for example SNPs are usually preceded and followed by highly conserved sequences that vary in less than 1/100 or 1/1000 members of the population. An individual may be homozygous or heterozygous for an allele at each SNP position. A SNP may, in some instances, be referred to as a “cSNP” to denote that the nucleotide sequence containing the SNP is an amino acid “coding” sequence. A SNP may arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa.

A synonymous codon change, or silent mutation is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. An indel that occur in a coding DNA segment gives rise to a frameshift mutation.

Causative variants are those that produce alterations in gene expression or in the structure and/or function of a gene product, and therefore are predictive of a possible clinical phenotype. One such class includes SNPs falling within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g., genetic endometriosis.

An association study of a variant and a specific disorder involves determining the presence or frequency of the variant allele in biological samples from individuals with the disorder of interest, such as endometriosis, and comparing the information to that of controls (i.e., individuals who do not have the disorder; controls may be also referred to as “healthy” or “normal” individuals) who are for example of similar age and race. The appropriate selection of patients and controls is important to the success of variant association studies. Therefore, a pool of individuals with well-characterized phenotypes is extremely desirable.

A variant may be screened in tissue samples or any biological sample obtained from an affected individual, and compared to control samples, and selected for its increased (or decreased) occurrence in a specific pathological condition, such as pathologies related to endometriosis. Once a statistically significant association is established between one or more variant(s) and a pathological condition (or other phenotype) of interest, then the region around the variant can optionally be thoroughly screened to identify the causative genetic locus/sequence(s) (e.g., causative variant/mutation, gene, regulatory region, etc.) that influences the pathological condition or phenotype. Association studies may be conducted within the general population and are not limited to studies performed on related individuals in affected families (linkage studies). For diagnostic and prognostic purposes, if a particular variant site is found to be useful for diagnosing a disease, such as endometriosis, other variant sites which are in LD with this variant site may also be expected to be useful for diagnosing the condition. Linkage disequilibrium is described in the human genome as blocks of variants along a chromosome segment that do not segregate independently (i.e., that are non-randomly co-inherited). The starting (5′ end) and ending (3′ end) of these blocks can vary depending on the criteria used for linkage disequilibrium in a given database, such as the value of D′ or r² used to determine linkage disequilibrium.

In some instances, variants can be identified in a study using a whole-genome case-control approach to identify single nucleotide polymorphisms that were closely associated with the development of endometriosis, as well as variants found to be in linkage disequilibrium with (i.e., within the same linkage disequilibrium block as) the endometriosis-associated variants, which can provide haplotypes (i.e., groups of variants that are co-inherited) to be readily inferred. Thus, the present disclosure provides individual variants associated with endometriosis, as well as combinations of variants and haplotypes in genetic regions associated with endometriosis, methods of detecting these polymorphisms in a test sample, methods of determining the risk of an individual of having or developing endometriosis and for clinical sub-classification of endometriosis.

In some cases, the present disclosure provides variants associated with endometriosis, as well as variants that were previously known in the art, but were not previously known to be associated with endometriosis. Accordingly, the present disclosure provides novel compositions and methods based on the variants disclosed herein, and also provides novel methods of using the known but previously unassociated variants in methods relating to endometriosis (e.g., for diagnosing endometriosis, etc.).

In some instances, particular variant alleles of the present disclosure can be associated with either an increased risk of having or developing endometriosis, or a decreased risk of having or developing endometriosis. Variant alleles that are associated with a decreased risk may be referred to as “protective” alleles, and variant alleles that are associated with an increased risk may be referred to as “susceptibility” alleles, “risk factors”, or “high-risk” alleles. Thus, whereas certain variants can be assayed to determine whether an individual possesses a variant allele that is indicative of an increased risk of having or developing endometriosis (i.e., a susceptibility allele), other variants can be assayed to determine whether an individual possesses a variant allele that is indicative of a decreased risk of having or developing endometriosis (i.e., a protective allele). Similarly, particular variant alleles of the present disclosure can be associated with either an increased or decreased likelihood of responding to a particular treatment. The term “altered” may be used herein to encompass either of these two possibilities (e.g., an increased or a decreased risk/likelihood).

In some instances, nucleic acid molecules may be double-stranded molecules and that reference to a particular site on one strand refers, as well, to the corresponding site on a complementary strand. In defining a variant position, variant allele, or nucleotide sequence, reference to an adenine, a thymine (uridine), a cytosine, or a guanine at a particular site on one strand of a nucleic acid molecule also defines the complementary thymine (uridine), adenine, guanine, or cytosine (respectively) at the corresponding site on a complementary strand of the nucleic acid molecule. Thus, reference may be made to either strand in order to refer to a particular variant position, variant allele, or nucleotide sequence. Probes and primers may be designed to hybridize to either strand and variant genotyping methods disclosed herein may generally target either strand. Throughout the specification, in identifying a variant position, reference is generally made to the forward or “sense” strand, solely for the purpose of convenience. Since endogenous nucleic acid sequences exist in the form of a double helix (a duplex comprising two complementary nucleic acid strands), it is understood that the variants disclosed herein will have counterpart nucleic acid sequences and variants associated with the complementary “reverse” or “antisense” nucleic acid strand. Such complementary nucleic acid sequences, and the complementary variants present in those sequences, are also included within the scope of the present disclosure.

Disclosed herein may be methods for detecting genetic variants in a nucleic acid sample. The method can comprise sequencing a nucleic acid sample obtained from a subject having endometriosis or suspected of having endometriosis using a high throughput method. The high throughput method can comprise nanopore sequencing. The method can comprise detecting one or more genetic variants in a nucleic acid sample, wherein the one or more genetic variants may be listed in Table 1, Table 2, or a combination thereof. The nucleic acid sample can comprise RNA. The RNA can comprise mRNA. The RNA can comprise cell-free RNA. The RNA can comprise miRNA, The nucleic acid sample can comprise DNA. The DNA can comprise cDNA, genomic DNA, sheared DNA, cell free DNA, fragmented DNA, or PCR amplified products produced therefrom, or any combination thereof. The one or more genetic variants can comprise a genetic variant defining a minor allele, an alternative allele, or a non-reference allele. The one or more genetic variants can comprise at least about: 5, 10, 15, 20, 25, 50, 75, 100, 150, 200, 250, 500, or more genetic variants defining minor alleles, alternative alleles, or non-reference alleles. The detection of the one or more genetic variants can have an odds ratio (OR) for endometriosis of at least about: 1.5, 2, 5, 10, 20, 50, 100, or more. The one or more genetic variants can comprise a synonymous mutation, a non-synonymous mutation, a stop-gain mutation, a nonsense mutation, an insertion, a deletion, a splice-site variant, a frameshift mutation, or any combination thereof. The one or more genetic variants can comprise a protein damaging mutation. The one or more genetic variants can be identified based on a predictive computer algorithm. The one or more genetic variants can be identified based on reference to a database. The method can further comprise identifying a subject as having endometriosis or being at risk of developing endometriosis. The method can comprise identifying a subject as having endometriosis or being at risk of developing endometriosis with a specificity of at least about: 80%, 85%, 90%, 95%, 96%, 97%, 98%, or 99%. The method can comprise identifying a subject as having endometriosis or being at risk of developing endometriosis with a sensitivity of at least about: 80%, 85%, 90%, 95%, 96%, 97%, 98%, or 99%. The method can comprise identifying a subject as having endometriosis or being at risk of developing endometriosis with an accuracy of at least about: 80%, 85%, 90%, 95%, 96%, 97%, 98%, or 99%. The method can comprise identifying a subject as having endometriosis. The subject can be asymptomatic for endometriosis. In some embodiments, the subject can have endometriosis and be asymptomatic. The subject can be symptomatic for endometriosis. The subject can be identified as being at risk of developing endometriosis. The method can further comprise administering a therapeutic to a subject. The therapeutic can comprise a pain medication. The pain medication can comprise a nonsteroidal anti-inflammatory drug (NSAID), ibuprofen, naproxen, an opioid, a cannabis-based therapeutic, or any combination thereof. In some embodiments, the one or more genetic variants may be listed in Table 1, Table 2, or a combination thereof. A subject described herein can be a mammal. The mammal can be a human. Nanopore sequencing can be performed with a biological nanopore, a solid state nanopore, or a hybrid nanopore. Methods can detect about: 1, 5, 10, 15, 20, 30, 50, 60, 100, 80, 90, 100, 200 or more variants. Genetic variants detected herein can indicate endometriosis or a risk of developing endometriosis. In some embodiments, one or more genetic variant listed in Table 1, Table 2, or a combination thereof may be the only genetic variants detected.

Genotyping Methods

In some cases, the process of determining which specific nucleotide (i.e., allele) is present at each of one or more variant positions, such as a variant position in a nucleic acid molecule characterized by a variant, is referred to as variant genotyping. The present disclosure provides methods of variant genotyping, such as for use in screening for endometriosis or related pathologies, or determining predisposition thereto, or determining responsiveness to a form of treatment, or in genome mapping or variant association analysis, etc.

Nucleic acid samples can be genotyped to determine which allele(s) is/are present at any given genetic region (e.g., variant position) of interest by methods well known in the art. The neighboring sequence can be used to design variant detection reagents such as oligonucleotide probes, which may optionally be implemented in a kit format. Variant genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, mass spectrometry with or with monoisotopic dNTPs (pyrosequencing, multiplex primer extension sorted on genetic arrays, ligation with rolling circle amplification, homogeneous ligation, OLA, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, electrospray mass spectrometry, and electrical detection.

Various methods for detecting polymorphisms can include, but are not limited to, methods in which protection from cleavage agents is used to detect mismatched bases in RNA/RNA or RNA/DNA duplexes, comparison of the electrophoretic mobility of variant and wild type nucleic acid molecules, and assaying the movement of polymorphic or wild-type fragments in polyacrylamide gels containing a gradient of denaturant using denaturing gradient gel electrophoresis (DGGE). Sequence variations at specific locations can also be assessed by nuclease protection assays such as RNase and SI protection or chemical cleavage methods.

In some instances, a variant genotyping can be performed using the TaqMan assay, which is also known as the 5′ nuclease assay. The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced. During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target variant-containing template which is amplified during PCR, and the probe is designed to hybridize to the target variant site only if a particular variant allele is present. TaqMan primer and probe sequences can readily be determined using the variant and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the variants of the present disclosure are useful in diagnostic assays for endometriosis and related pathologies, and can be readily incorporated into a kit format. The present disclosure also includes modifications of the Taqman assay well known in the art such as the use of Molecular Beacon probes and other variant formats.

In some instances, a method for genotyping the variants can be the use of two oligonucleotide probes in an OLA. In this method, one probe hybridizes to a segment of a target nucleic acid with its 3′ most end aligned with the variant site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the variant site. If there is a mismatch, ligation may not occur. After the reaction, the ligated probes are separated from the target nucleic acid molecule, and detected as indicators of the presence of a variant.

In some instances, a method for variant genotyping is based on mass spectrometry. Mass spectrometry takes advantage of the unique mass of each of the four nucleotides of DNA. Variants can be unambiguously genotyped by mass spectrometry by measuring the differences in the mass of nucleic acids having alternative variant alleles. MALDI-TOF (Matrix Assisted Laser Desorption Ionization-Time of Flight) mass spectrometry technology is exemplary for extremely precise determinations of molecular mass, such as variants. Numerous approaches to variant analysis have been developed based on mass spectrometry. Exemplary mass spectrometry-based methods of variant genotyping include primer extension assays, which can also be utilized in combination with other approaches, such as traditional gel-based formats and microarrays.

In some instances, a method for genotyping the variants of the present disclosure is the use of electrospray mass spectrometry for direct analysis of an amplified nucleic acid. In this method, in one aspect, an amplified nucleic acid product may be isotopically enriched in an isotope of oxygen (O), carbon (C), nitrogen (N) or any combination of those elements. In an exemplary embodiment the amplified nucleic acid is isotopically enriched to a level of greater than 99.9% in the elements of O¹⁶, C¹² and N¹⁴. The amplified isotopically enriched product can then be analyzed by electrospray mass spectrometry to determine the nucleic acid composition and the corresponding variant genotyping. Isotopically enriched amplified products result in a corresponding increase in sensitivity and accuracy in the mass spectrum. In another aspect of this method an amplified nucleic acid that is not isotopically enriched can also have composition and variant genotype determined by electrospray mass spectrometry.

In some instances, variants can be scored by direct DNA sequencing. The nucleic acid sequences of the present disclosure enable one of ordinary skill in the art to readily design sequencing primers for such automated sequencing procedures. Commercial instrumentation, such as the Applied Biosystems 377, 3100, 3700, 3730, and 3730.times.1 DNA Analyzers (Foster City, Calif.), may be used for automated sequencing.

Variant genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target variant under conditions such that hybridization and amplification of the target nucleic acid region occurs, and determining the nucleotide present at the variant position of interest, or, in some assays, detecting the presence or absence of an amplification product (assays can be designed so that hybridization and/or amplification will only occur if a particular variant allele is present or absent). In some assays, the size of the amplification product is detected and compared to the length of a control sample; for example, deletions and insertions can be detected by a change in size of the amplified product compared to a normal genotype.

In some instances, a variant genotyping can be used in applications that include, but are not limited to, variant-endometriosis association analysis, endometriosis predisposition screening, endometriosis diagnosis, endometriosis prognosis, endometriosis progression monitoring, determining therapeutic strategies based on an individual's genotype, and stratifying a patient population for clinical trials for a treatment such as minimally invasive device for the treatment of endometriosis.

Analysis of Genetic Association Between Variants and Phenotypic Traits

In some cases, genotyping for endometriosis diagnosis, endometriosis predisposition screening, endometriosis prognosis and endometriosis treatment and other uses described herein, can rely on initially establishing a genetic association between one or more specific variants and the particular phenotypic traits of interest.

In some instances, in a genetic association study, the cause of interest to be tested is a certain allele or a variant or a combination of alleles or a haplotype from several variants. Thus, tissue specimens (e.g., saliva) from the sampled individuals may be collected and genomic DNA genotyped for the variant(s) of interest. In addition to the phenotypic trait of interest, other information such as demographic (e.g., age, gender, ethnicity, etc.), clinical, and environmental information that may influence the outcome of the trait can be collected to further characterize and define the sample set. Specifically, in an endometriosis genetic association study, clinical information such as body mass index, age and diet may be collected. In many cases, these factors are known to be associated with diseases and/or variant allele frequencies. There are likely gene-environment and/or gene-gene interactions as well. Analysis methods to address gene-environment and gene-gene interactions (for example, the effects of the presence of both susceptibility alleles at two different genes can be greater than the effects of the individual alleles at two genes combined) are discussed below.

In some instances, after all the relevant phenotypic and genotypic information has been obtained, statistical analyses are carried out to determine if there is any significant correlation between the presence of an allele or a genotype with the phenotypic characteristics of an individual. For example, data inspection and cleaning are first performed before carrying out statistical tests for genetic association. Epidemiological and clinical data of the samples can be summarized by descriptive statistics with tables and graphs. Data validation is for example performed to check for data completion, inconsistent entries, and outliers. Chi-squared tests may then be used to check for significant differences between cases and controls for discrete and continuous variables, respectively. To ensure genotyping quality, Hardy-Weinberg disequilibrium tests can be performed on cases and controls separately. Significant deviation from Hardy-Weinberg equilibrium (HWE) in both cases and controls for individual markers can be indicative of genotyping errors. If HWE is violated in a majority of markers, it is indicative of population substructure that may be further investigated. Moreover, Hardy-Weinberg disequilibrium in cases only can indicate genetic association of the markers with the disease of interest.

In some instances, to test whether an allele of a single variant is associated with the case or control status of a phenotypic trait, one skilled in the art can compare allele frequencies in cases and controls. Standard chi-squared tests and Fisher exact tests can be carried out on a 2.times.2 table (2 variant alleles.times.2 outcomes in the categorical trait of interest). To test whether genotypes of a variant are associated, chi-squared tests can be carried out on a 3.times.2 table (3 genotypes.times.2 outcomes). Score tests are also carried out for genotypic association to contrast the three genotypic frequencies (major homozygotes, heterozygotes and minor homozygotes) in cases and controls, and to look for trends using 3 different modes of inheritance, namely dominant (with contrast coefficients 2, −1, −1), additive (with contrast coefficients 1, 0, −1) and recessive (with contrast coefficients 1, 1, −2). Odds ratios for minor versus major alleles, and odds ratios for heterozygote and homozygote variants versus the wild type genotypes are calculated with the desired confidence limits, usually 95%. In the present study a software algorithm, PLINK, has been applied to automate the calculation of Hardy-Weinberg equilibrium, chi-square, p-values and odds-ratios for very large numbers of variants and Case-Control individuals simultaneously.

In some instances, in order to control for confounding effects and to test for interactions a stepwise multiple logistic regression analysis using statistical packages such as SAS or R may be performed. Logistic regression is a model-building technique in which the best fitting and most parsimonious model is built to describe the relation between the dichotomous outcome (for instance, getting a certain endometriosis or not) and a set of independent variables (for instance, genotypes of different associated genes, and the associated demographic and environmental factors). A model may include one in which the logit transformation of the odds ratios is expressed as a linear combination of the variables (main effects) and their cross-product terms (interactions). To test whether a certain variable or interaction is significantly associated with the outcome, coefficients in the model are first estimated and then tested for statistical significance of their departure from zero.

In some instances, in addition to performing association tests one marker at a time, haplotype association analysis may also be performed to study a number of markers that are closely linked together. Haplotype association tests can have better power than genotypic or allelic association tests when the tested markers are not the disease-causing mutations themselves but are in linkage disequilibrium with such mutations. The test will even be more powerful if the endometriosis is indeed caused by a combination of alleles on a haplotype. In order to perform haplotype association effectively, marker-marker linkage disequilibrium measures, both D′ and r², may be calculated for the markers within a gene to elucidate the haplotype structure. Variants within a gene can be organized in block pattern, and a high degree of linkage disequilibrium exists within blocks and very little linkage disequilibrium exists between blocks. Haplotype association with the endometriosis status can be performed using such blocks once they have been elucidated.

Haplotype association tests can be carried out in a similar fashion as the allelic and genotypic association tests. Each haplotype in a gene is analogous to an allele in a multi-allelic marker. One skilled in the art can either compare the haplotype frequencies in cases and controls or test genetic association with different pairs of haplotypes. Score tests can be done on haplotypes using the program “haplo.score”. In that method, haplotypes are first inferred by EM algorithm and score tests are carried out with a generalized linear model (GLM) framework that allows the adjustment of other factors.

In some instances, an important decision in the performance of genetic association tests is the determination of the significance level at which significant association can be declared when the p-value of the tests reaches that level. In an exploratory analysis where positive hits will be followed up in subsequent confirmatory testing, an unadjusted p-value <0.1 (a significance level on the lenient side) may be used for generating hypotheses for significant association of a variant with certain phenotypic characteristics of a endometriosis. It is exemplary that a p-value <0.05 (a significance level traditionally used in the art) is achieved in order for a variant to be considered to have an association with a endometriosis. It is more exemplary that a p-value <0.01 (a significance level on the stringent side) is achieved for an association to be declared. Permutation tests to control for the false discovery rates, FDR, can further be employed. Such methods to control for multiplicity may be exemplary when the tests are dependent and controlling for false discovery rates is sufficient as opposed to controlling for the experiment-wise error rates.

In some instances, since both genotyping and endometriosis status classification can involve errors, sensitivity analyses may be performed to see how odds ratios and p-values may change upon various estimates on genotyping and endometriosis classification error rates.

Once individual risk factors, genetic or non-genetic, have been found for the predisposition to endometriosis, the next step can be to set up a classification/prediction scheme to predict the category (for instance, endometriosis or no endometriosis) that an individual will be in depending on his genotypes of associated variants and other non-genetic risk factors. Logistic regression for discrete trait and linear regression for continuous trait are standard techniques for such tasks. Moreover, other techniques can also be used for setting up classification. Such techniques include, but are not limited to, MART, CART, neural network, and discriminant analyses that are suitable for use in comparing the performance of different methods.

Endometriosis Diagnosis and Predisposition Screening

In some cases, information on association/correlation between genotypes and endometriosis-related phenotypes can be exploited in several ways. For example, in the case of a highly statistically significant association between one or more variants with predisposition to a disease for which treatment is available, detection of such a genotype pattern in an individual may justify particular treatment, or at least the institution of regular monitoring of the individual. In the case of a weaker but still statistically significant association between a variant and a human disease, immediate therapeutic intervention or monitoring may not be justified after detecting the susceptibility allele or variant.

The variants disclosed herein may contribute to endometriosis in an individual in different ways. Some polymorphisms occur within a protein coding sequence and contribute to endometriosis phenotype by affecting protein structure. Other polymorphisms occur in noncoding regions but may exert phenotypic effects indirectly via influence on, for example, replication, transcription, and/or translation. A single variant may affect more than one phenotypic trait. Likewise, a single phenotypic trait may be affected by multiple variants in different genes.

The variants disclosed herein may contribute to endometriosis in an individual in different ways. Some polymorphisms occur within a protein coding sequence and contribute to endometriosis phenotype by affecting protein structure. Other polymorphisms occur in noncoding regions but may exert phenotypic effects indirectly via influence on, for example, replication, transcription, and/or translation. A single variant may affect more than one phenotypic trait. Likewise, a single phenotypic trait may be affected by multiple variants in different genes.

Haplotypes can be particularly useful in that, for example, fewer variants can be genotyped to determine if a particular genomic region harbors a locus that influences a particular phenotype, such as in linkage disequilibrium-based variant association analysis.

Linkage disequilibrium (LD) can refer to the co-inheritance of alleles (e.g., alternative nucleotides) at two or more different variant sites at frequencies greater than may be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium”. In contrast, LD refers to any non-random genetic association between allele(s) at two or more different variant sites, which is generally due to the physical proximity of the two loci along a chromosome. LD can occur when two or more variants sites are in close physical proximity to each other on a given chromosome and therefore alleles at these variant sites will tend to remain unseparated for multiple generations with the consequence that a particular nucleotide (allele) at one variant site will show a non-random association with a particular nucleotide (allele) at a different variant site located nearby. Hence, genotyping one of the variant sites will give almost the same information as genotyping the other variant site that is in LD.

For diagnostic purposes, if a particular variant site is found to be useful for diagnosing endometriosis, then the skilled artisan may recognize that other variant sites which are in LD with this variant site may also be useful for diagnosing the condition. Various degrees of LD can be encountered between two or more variants with the result being that some variants are more closely associated (i.e., in stronger LD) than others. Furthermore, the physical distance over which LD extends along a chromosome differs between different regions of the genome, and therefore the degree of physical separation between two or more variant sites necessary for LD to occur can differ between different regions of the genome.

For diagnostic applications, polymorphisms (e.g., variants and/or haplotypes) that are not the actual disease-causing (causative) polymorphisms, but are in LD with such causative polymorphisms, are also useful. In such instances, the genotype of the polymorphism(s) that is/are in LD with the causative polymorphism is predictive of the genotype of the causative polymorphism and, consequently, predictive of the phenotype (e.g., endometriosis) that is influenced by the causative variant(s). Thus, polymorphic markers that are in LD with causative polymorphisms are useful as diagnostic markers, and are particularly useful when the actual causative polymorphism(s) is/are unknown.

The contribution or association of particular variants and/or variant haplotypes with endometriosis phenotypes, such as endometriosis, can enable the variants of the present disclosure to be used to develop superior diagnostic tests capable of identifying individuals who express a detectable trait, such as endometriosis, as the result of a specific genotype, or individuals whose genotype places them at an increased or decreased risk of developing a detectable trait at a subsequent time as compared to individuals who do not have that genotype. As described herein, diagnostics may be based on a single variant or a group of variants. In some instances, combined detection of a plurality of variations, for example about 2, 3, 4, 5, 6, 7, 8, 9, 10, 11, 12, 13, 14, 15, 16, 17, 18, 19, 20, 24, 25, 30, 32, 35, 40, 45, 48, 50, 55, 60, 64, 70, 75, 80, 85, 80, 96, 100, or any other number in-between, or more, of the variants provided herein can increase the probability of an accurate diagnosis. To further increase the accuracy of diagnosis or predisposition screening, analysis of the variants of the present disclosure can be combined with that of other polymorphisms or other risk factors of endometriosis, such as gender and age.

In some instances, the method herein can indicate a certain increased (or decreased) degree or likelihood of developing the endometriosis based on statistically significant association results. This information can be valuable to initiate earlier preventive treatments or to allow an individual carrying one or more significant variants or variant haplotypes to regularly scheduled physical exams to monitor for the appearance or change of their endometriosis in order to identify and begin treatment of the endometriosis at an early stage.

The diagnostic techniques herein may employ a variety of methodologies to determine whether a test subject has a variant or a variant pattern associated with an increased or decreased risk of developing a detectable trait or whether the individual suffers from a detectable trait as a result of a particular polymorphism/mutation, including, for example, methods which enable the analysis of individual chromosomes for haplotyping, family studies, single sperm DNA analysis, or somatic hybrids. The trait analyzed using the diagnostics of the disclosure may be any detectable trait that is observed in pathologies and disorders related to endometriosis.

Another aspect of the present disclosure relates to a method of determining whether an individual is at risk (or less at risk) of developing one or more traits or whether an individual expresses one or more traits as a consequence of possessing a particular trait-causing or trait-influencing allele. These methods generally involve obtaining a nucleic acid sample from an individual and assaying the nucleic acid sample to determine which nucleotide(s) is/are present at one or more variant positions, wherein the assayed nucleotide(s) is/are indicative of an increased or decreased risk of developing the trait or indicative that the individual expresses the trait as a result of possessing a particular trait-causing or trait-influencing allele.

The variants herein can be used to identify novel therapeutic targets for endometriosis. For example, genes containing the disease-associated variants (“variant genes”) or their products, as well as genes or their products that are directly or indirectly regulated by or interacting with these variant genes or their products, can be targeted for the development of therapeutics that, for example, treat the endometriosis or prevent or delay endometriosis onset. The therapeutics may be composed of, for example, small molecules, proteins, protein fragments or peptides, antibodies, nucleic acids, or their derivatives or mimetics which modulate the functions or levels of the target genes or gene products.

The variants/haplotypes herein can be useful for improving many different aspects of the drug development process. For example, individuals can be selected for clinical trials based on their variant genotype. Individuals with variant genotypes that indicate that they are most likely to respond to or most likely to benefit from a device or a drug can be included in the trials and those individuals whose variant genotypes indicate that they are less likely to or may not respond to a device or a drug, or suffer adverse reactions, can be eliminated from the clinical trials. This not only improves the safety of clinical trials, but also will enhance the chances that the trial will demonstrate statistically significant efficacy. Furthermore, the variants of the present disclosure may explain why certain previously developed devices or drugs performed poorly in clinical trials and may help identify a subset of the population that may benefit from a drug that had previously performed poorly in clinical trials, thereby “rescuing” previously developed therapeutic treatment methods or drugs, and enabling the methods or drug to be made available to a particular endometriosis patient population that can benefit from it.

Detection Kits and Systems

In some instances, based on a variant such as SNP or indels and associated sequence information disclosed herein, detection reagents can be developed and used to assay any variant of the present disclosure individually or in combination, and such detection reagents can be readily incorporated into one of the established kit or system formats which are well known in the art. The terms “kits” and “systems” can refer to such things as combinations of multiple variant detection reagents, or one or more variant detection reagents in combination with one or more other types of elements or components (e.g., other types of biochemical reagents, containers, packages such as packaging intended for commercial sale, substrates to which variant detection reagents are attached, electronic hardware components, etc.). Accordingly, the present disclosure further provides variant detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more variants of the present disclosure. The kits/systems can optionally include various electronic hardware components; for example, arrays (“DNA chips”) and microfluidic systems (“lab-on-a-chip” systems) provided by various manufacturers may comprise hardware components. Other kits/systems (e.g., probe/primer sets) may not include electronic hardware components, but may be comprised of, for example, one or more variant detection reagents (along with, optionally, other biochemical reagents) packaged in one or more containers.

In some instances, provided herein is a kit comprising one or more variant detection agents, and methods for detecting the variants disclosed herein by employing detection reagents and optionally a questionnaire of non-genetic clinical factors. In some instances, provided herein is a method of identifying an individual having an increased or decreased risk of developing endometriosis by detecting the presence or absence of a variant allele disclosed herein. In some instances, provided herein is a method for diagnosis of endometriosis by detecting the presence or absence of a variant allele disclosed herein is provided. In some instances, provided herein is a method for predicting endometriosis sub-classification by detecting the presence or absence of a variant allele. In some instances, the questionnaire may be completed by a medical professional based on medical history physical exam or other clinical findings. In some instances, the questionnaire may include any other non-genetic clinical factors known to be associated with the risk of developing endometriosis. In some instances, a reagent for detecting a variant in the context of its naturally-occurring flanking nucleotide sequences (which can be, e.g., either DNA or mRNA) is provided. In some instances, the reagent may be in the form of a hybridization probe or an amplification primer that is useful in the specific detection of a variant of interest. In some instances, a variant can be a genetic polymorphism having a Minor Allele Frequency (MAF) of at least 1% in a population (such as for instance the Caucasian population or the CEU population) and an RV is understood to be a genetic polymorphism having a Minor Allele Frequency (MAF) of less than 1% in a population (such as for instance the Caucasian population or the CEU population).

In some instances, a detection kit can contain one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a variant-containing nucleic acid molecule. A kit may further contain means for determining the amount of a target nucleic acid, and means for comparing the amount with a standard, and can comprise instructions for using the kit to detect the variant-containing nucleic acid molecule of interest. In one embodiment of the present disclosure, kits are provided which contain the necessary reagents to carry out one or more assays to detect one or more variants disclosed herein. In an exemplary embodiment of the present disclosure, the detection kits/systems can be in the form of nucleic acid arrays, or compartmentalized kits, including microfluidic/lab-on-a-chip systems.

In some instances, variant detection kits/systems may contain, for example, one or more probes, or pairs of probes, that hybridize to a nucleic acid molecule at or near each target variant position. Multiple pairs of allele-specific probes may be included in the kit/system to simultaneously assay large numbers of variants, at least one of which is a variant of the present disclosure. In some kits/systems, the allele-specific probes are immobilized to a substrate such as an array or bead. For example, the same substrate can comprise allele-specific probes for detecting at least 1; 10; 100; 1000; 10,000; 100,000; 500,000 (or any other number in-between) or substantially all of the variants disclosed herein.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate.

In some instances, any number of probes, such as allele-specific probes, may be implemented in an array, and each probe or pair of probes can hybridize to a different variant position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). For example, probes are attached to a solid support in an ordered, addressable array.

In some instances, a microarray can be composed of a large number of unique, single-stranded polynucleotides fixed to a solid support. Polynucleotides may include for example about 6-60 nucleotides in length, more for example about 15-30 nucleotides in length, and most for example about 18-25 nucleotides in length. For certain types of microarrays or other detection kits/systems, it may be suitable to use oligonucleotides that are only about 7-20 nucleotides in length. In other types of arrays, such as arrays used in conjunction with chemiluminescent detection technology, exemplary probe lengths can be, for example, about 15-80 nucleotides in length, for example about 50-70 nucleotides in length, more for example about 55-65 nucleotides in length, and most for example about 60 nucleotides in length. The microarray or detection kit can contain polynucleotides that cover the known 5′ or 3′ sequence of the target variant site, sequential polynucleotides that cover the full-length sequence of a gene/transcript; or unique polynucleotides selected from particular areas along the length of a target gene/transcript sequence, particularly areas corresponding to one or more variants disclosed herein. Polynucleotides used in the microarray or detection kit can be specific to a variant or variants of interest (e.g., specific to a particular SNP allele at a target SNP site, or specific to particular SNP alleles at multiple different SNP sites), or specific to a polymorphic gene/transcript or genes/transcripts of interest.

In some instances, hybridization assays based on polynucleotide arrays rely on the differences in hybridization stability of the probes to perfectly matched and mismatched target sequence variants. For variant genotyping, it is generally suitable that stringency conditions used in hybridization assays are high enough such that nucleic acid molecules that differ from one another at as little as a single variant position can be differentiated (e.g., variant hybridization assays may be designed so that hybridization will occur only if one particular nucleotide is present at a variant position, but will not occur if an alternative nucleotide is present at that variant position). Such high stringency conditions may be suitable when using, for example, nucleic acid arrays of allele-specific probes for variant detection. In some instances, the arrays are used in conjunction with chemiluminescent detection technology.

In some instances, a nucleic acid array can comprise an array of probes of about 15-25 nucleotides in length. In further embodiments, a nucleic acid array can comprise any number of probes, in which at least one probe is capable of detecting one or more variants disclosed herein and/or at least one probe comprises a fragment of one of the sequences selected from the group consisting of those disclosed herein, and sequences complementary thereto, said fragment comprising at least about 8 consecutive nucleotides, for example 10, 12, 15, 16, 18, 20, more for example 22, 25, 30, 40, 47, 50, 55, 60, 65, 70, 80, 90, 100, or more consecutive nucleotides (or any other number in-between) and containing (or being complementary to) a variant. In some embodiments, the nucleotide complementary to the variant site is within 5, 4, 3, 2, or 1 nucleotide from the center of the probe, more for example at the center of said probe.

In some instances, using such arrays or other kits/systems, the present disclosure provides methods of identifying the variants disclosed herein in a test sample. Such methods may involve incubating a test sample of nucleic acids with an array comprising one or more probes corresponding to at least one variant position of the present disclosure, and assaying for binding of a nucleic acid from the test sample with one or more of the probes. Conditions for incubating a variant detection reagent (or a kit/system that employs one or more such variant detection reagents) with a test sample vary. Incubation conditions depend on such factors as the format employed in the assay, the detection methods employed, and the type and nature of the detection reagents used in the assay. One skilled in the art will recognize that any number of available hybridization, amplification and array assay formats can readily be adapted to detect the variants disclosed herein.

In some instances, a detection kit/system may include components that are used to prepare nucleic acids from a test sample for the subsequent amplification and/or detection of a variant-containing nucleic acid molecule. Such sample preparation components can be used to produce nucleic acid extracts, including DNA and/or RNA, extracts from any bodily fluids. In an exemplary embodiment of the disclosure, the bodily fluid is blood, saliva or buccal swabs. The test samples used in the above-described methods will vary based on such factors as the assay format, nature of the detection method, and the specific tissues, cells or extracts used as the test sample to be assayed. Methods of preparing nucleic acids are well known in the art and can be readily adapted to obtain a sample that is compatible with the system utilized. In some instances, in addition to reagents for preparation of nucleic acids and reagents for detection of one of the variants of this disclosure, the kit may include a questionnaire inquiring about non-genetic clinical factors such as age, gender, or any other non-genetic clinical factors known to be associated with endometriosis.

In some instances, a form of kit can be a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other variant detection reagent for detecting one or more variants of the present disclosure, one or more containers which contain wash reagents (such as phosphate buffered saline, Tris-buffers, etc.), and one or more containers which contain the reagents used to reveal the presence of the bound probe or other variant detection reagents. The kit can optionally further comprise compartments and/or reagents for, for example, nucleic acid amplification or other enzymatic reactions such as primer extension reactions, hybridization, ligation, electrophoresis (for example capillary electrophoresis), mass spectrometry, and/or laser-induced fluorescent detection. The kit may also include instructions for using the kit. In such microfluidic devices, the containers may be referred to as, for example, microfluidic “compartments”, “chambers”, or “channels”.

In some instances, microfluidic devices, which may also be referred to as “lab-on-a-chip” systems, biomedical micro-electro-mechanical systems (bioMEMs), or multicomponent integrated systems, are exemplary kits/systems of the present disclosure for analyzing variants. Such systems miniaturize and compartmentalize processes such as probe/target hybridization, nucleic acid amplification, and capillary electrophoresis reactions in a single functional device. Such microfluidic devices may utilize detection reagents in at least one aspect of the system, and such detection reagents may be used to detect one or more variants of the present disclosure. One example of a microfluidic system is the integration of PCR amplification and capillary electrophoresis in chips. Exemplary microfluidic systems comprise a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. In some instances, for genotyping variants, a microfluidic system may integrate, for example, nucleic acid amplification, primer extension, capillary electrophoresis, and a detection method such as laser induced fluorescence detection.

Methods of Treatment

In some aspects, disclosed herein is a method of treating a select subject in need thereof. The use of these genetic markers can allow selection of subjects for clinical trials involving novel treatment methods. In some cases, genetic markers disclosed herein can be used for early diagnosis and prognosis of endometriosis, as well as early clinical intervention to mitigate progression of the disease. In some instances, genetic markers disclosed herein can be used to predict endometriosis and endometriosis progression, for example in treatment decisions for individuals who are recognized as having endometriosis.

In some cases, a treatment disclosed herein includes one or more of: reducing the frequency and/or severity of symptoms, elimination of symptoms and/or their underlying cause, and improvement or remediation of damage. For example, treatment of endometriosis includes, relieving the pain experienced by a woman suffering from endometriosis, and/or causing the regression or disappearance of endometriotic lesions.

In some cases, the treatment can be an advanced reproductive therapy such as in vitro in fertilization (IVF); a hormonal treatment; progestogen; progestin; an oral contraceptive; a hormonal contraceptive; danocrine; gentrinone; a gonadotrophin releasing hormone agonist; Lupron; danazol; an aromatase inhibitor; pentoxifylline; surgical treatment; laparoscopy; cauterization; or cystectomy. In some instances, the progestogen can be progesterone, desogestrel, etonogestrel, gestodene, levonorgestrel, medroxyprogesterone, norethisterone, norgestimate, megestrol, megestrol acetate, norgestrel, a pharmaceutically acceptable salt thereof (e.g., acetate), or any combination thereof. In some instances, a therapeutic used herein is selected from progestins, estrogens, antiestrogens, and antiprogestins, for example micronized danazol in a micro- or nanoparticulate formulation.

In some cases, a method of treatment disclosed herein comprises direct administration into or within an endometriotic lesion in a subject suffering from endometriosis of a pharmaceutical composition comprising a therapeutic disclosed herein. In some instances, the therapeutic is micronized in a suspension, e.g., non-oil based suspension. In some embodiments, the suspension comprises water, sodium sulfate, a quaternary ammonium wetting agent, glycerol, propylene glycol, polyethylene glycol, polypropylene glycol, a hydrophilic colloid, or any combination thereof.

The term “effective amount,” as used herein, can refer to a sufficient amount of a therapeutic being administered which relieve to some extent one or more of the symptoms of the disease or condition being treated. The result can be reduction and/or alleviation of the signs, symptoms, or causes of a disease, or any other desired alteration of a biological system. A therapeutic can be administered for prophylactic, enhancing, and/or therapeutic treatments. An appropriate “effective” amount in any individual case can be determined using techniques, such as a dose escalation study.

A treatment can comprise administering a therapeutic to a subject, intralesionally, transvaginally, intravenously, subcutaneously, intramuscularly, by inhalation, dermally, intra-articular injection, orally, intrathecally, transdermally, intranasally, via a peritoneal route, or directly onto or into a lesion/site, e.g., via endoscopically, open surgical administration, or injection route of application. In some instances, intralesional administration can mean administration into or within a pathological area. Administration can be effected by injection into a lesion and/or by instillation into a pre-existing cavity, such as in endometrioma. With reference to treatments for endometriosis provided herein, intralesional administration can refer to treatment within endometriotic tissue or a cyst formed by such tissue, such as by injection into a cyst. In some instances, intralesional administration can include administration into tissue in such close proximity to the endometriotic tissue such that the progestogen acts directly on the endometriotic tissue. In some instances, intralesional administration may or may not include administration to tissue remote from the endometriotic tissue that the progestogen acts on the endometriotic tissue through systemic circulation. In some instances, intralesional administration or delivery includes transvaginal, endoscopic or open surgical administration including, but are not limited to, via laparotomy. In some instances, transvaginal administration can refer to all procedures, including drug delivery, performed through the vagina, including intravaginal delivery and transvaginal sonography (ultrasonography through the vagina).

In some instances, administration is by injection into the endometriotic tissue or into a cyst formed by such tissue; or into tissue immediately surrounding the endometriotic tissue in such proximity that the progestogen acts directly on the endometriotic tissue. In some embodiments, the tissue is visualized, for example laparoscopically or by ultrasound, and the progestogen is administered by intralesional (intracystic) injection by, for example direct visualization under ultrasound guidance or by any other suitable methods. A suitable amount of the therapeutic, e.g., progestogen expressed in terms of progesterone of about 1-2 gm per lesion/cyst, can be applied. Precise quantity generally is determined on case to case basis, depending upon parameters, such as the size of the endometriotic tissue mass, the mode of the administration, and the number and time intervals between treatments.

In some instances, methods herein can comprise intralesional delivery of the medicaments into the lesion. Intralesional delivery includes, for example, transvaginal, endoscopic or open surgical administration including via laparotomy. Delivery can be effected, for example, through a needle or needle like device by injection or a similar injectable or syringe-like device that can be delivered into the lesion, such as transvaginally, endoscopically or by open surgical administration including via laparotomy. In some embodiments, the method includes intravaginal and transvaginal delivery. For intravaginal/transvaginal delivery an ultrasound probe can be used to guide delivery of the needle from the vagina into lesions such as endometriomas and utero sacral nodules. Under ultrasound guidance the needle tip is placed in the lesion, the contents of the lesion aspirated if necessary and the formulation is injected into the lesion. In an exemplary delivery system a 17 to 20 gauge needle can be used for injection of the drug. Such system can be used for intralesional delivery including, but not limited to, transvaginal, endoscopic or open surgical administration including via laparotomy. For treatment of endometrioma 17 or 18 gauge needles are used under ultrasound guidance for aspiration of the thick contents of the lesion and delivery of the formulation. The length of the needle used depends on the depth of the lesion. Pre-loaded syringes and other administration systems, which obviate the need for reloading the drug can be used.

In some cases, a therapeutic (e.g., an active agent) used herein can be a solution, a suspension, liquid, a paste, aqueous, non-aqueous fluid, semi-solids, colloid, gel, lotion, cream, solid (e.g., tablet, powder, pellet, particulate, capsule, packet), or any combination thereof. In some instances, a therapeutic disclosed herein is formulated as a dosage form of tablet, capsule, gel, lollipop, parenteral, intraspinal infusion, inhalation, spray, aerosol, transdermal patch, iontophoresis transport, absorbing gel, liquid, liquid tannate, suppositories, injection, I.V. drip, or a combination thereof to treat subjects. In some instances, the active agents are formulated as single oral dosage form such as a tablet, capsule, cachet, soft gelatin capsule, hard gelatin capsule, extended release capsule, tannate tablet, oral disintegrating tablet, multi-layer tablet, effervescent tablet, bead, liquid, oral suspension, chewable lozenge, oral solution, lozenge, lollipop, oral syrup, sterile packaged powder including pharmaceutically-acceptable excipients, other oral dosage forms, or a combination thereof. In some instances, a therapeutic of the disclosure herein can be administered using one or more different dosage forms which are further disclosed herein. In some instances, therapeutics disclosed herein are provided in modified release dosage forms (such as immediate release, controlled release, or both).

The methods, compositions, and kits of this disclosure can comprise a method to prevent, treat, arrest, reverse, or ameliorate the symptoms of a condition of a subject, e.g., a patient. A subject can be, for example, an elderly adult, adult, adolescent, pre-adolescence, teenager, or child. A subject can be, for example, 10-50 years old, 10-40 years old, 10-30 years old, 10-25 years old, 10-21 years old, 10-18 years old, 10-16 years old, 18-25 years old, or 16-34 years old. The subject can be a female mammal, e.g., a female human being. In some instances, the human subject can be asymptomatic for endometriosis.

Treatment can be provided to the subject before clinical onset of disease. Treatment can be provided to the subject after clinical onset of disease. Treatment can be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment can also include treating a human in a clinical trial.

A treatment, e.g., administration of a therapeutic, can occur 1, 2, 3, 4, 5, 6, 7, or 8 times daily. A treatment, e.g., administration of a therapeutic, can occur 1, 2, 3, 4, 5, 6, or 7 times weekly. A treatment, e.g., administration of a therapeutic, can occur 1, 2, 3, 4, 5, 6, 7, 8, 9, or 10 times monthly. A treatment, e.g., administration of a therapeutic, can occur 1, 2, 3, 4, 5, 6, 7, 8, 9, 10, 11, or 12 times yearly. In some instances, therapeutics disclosed herein are administered to a subject at about every 4 to about 6 hours, about every 12 hours, about every 24 hours, about every 48 hours, or more often. In some instances, therapeutics disclosed herein can be administered once, twice, three times, four times, five times, six times, seven times, eight times, or more often daily. In some instances, a dosage form disclosed herein provides an effective plasma concentration of an active agent at from about 1 minute to about 20 minutes after administration, such as about: 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min. In some instances, a dosage form of the disclosure herein provides an effective plasma concentration of an active agent at from about 20 minutes to about 24 hours after administration, such as about 20 minutes, 30 minutes, 40 minutes, 50 minutes, 1 hr, 1.2 hrs, 1.4 hrs, 1.6 hrs, 1.8 hrs, 2 hrs, 2.2 hrs, 2.4 hrs, 2.6 hrs, 2.8 hrs, 3 hrs, 3.2 hrs, 3.4 hrs, 3.6 hrs, 3.8 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, or 24 hrs following administration. In some instances, an active agent can be present in an effective plasma concentration in a subject for about 4 to about 6 hours, about 12 hours, about 24 hour, or 1 day to 30 days, including but not limited to 1, 2, 3, 4, 5, 6, 7, 8, 9, 10, 12, 12, 13, 14, 15, 16, 17, 18, 19, 20, 21, 22, 23, 24, 25, 26, 27, 28, 29 or 30 days.

In some instances, a therapeutic (e.g., an active agent) is administered to a subject in a dosage of about 0.01 mg to about 500 mg per day, e.g., about 1-50 mg/day for an average person. In some embodiments, the daily dosage is from about 0.01 mg to about 5 mg, about 1 to about 10 mg, about 5 mg to about 20 mg, about 10 mg to about 50 mg, about 20 mg to about 100 mg, about 50 mg to about 150 mg, about 100 mg to about 250 mg, about 150 mg to about 300 mg, or about 250 mg to about 500 mg.

In some instances, each administration of a therapeutic (e.g., an active agent) is in an amount of about: 0.1-5 mg, 0.1-10 mg, 1-5 mg, 1-10 mg, 1-20 mg, 10-20 mg, 10-30 mg, 10-40 mg, 10-50 mg, 20-30 mg, 20-40 mg, 20-50 mg, 25-50 mg, 30-40 mg, 30-50 mg, 30-60 mg, 40-50 mg, 40-60 mg, 50-60 mg, 50-75 mg, 60-80 mg, 75-100 mg, or 80-100 mg, for example: about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 22.5 mg, about 25 mg, about 27.5 mg, about 30 mg, about 32.5 mg, about 35 mg, about 37.5 mg, about 40 mg, about 42.5 mg, about 45 mg, about 47.5 mg, about 50 mg, about 55 mg, about 60 mg, about 65 mg, about 70 mg, about 75 mg, about 80 mg, about 85 mg, about 90 mg, about 95 mg, or about 100 mg.

In some instances, a therapeutic (e.g., an active agent) is administered to a subject in a dosage of about 0.01 g to about 100 g per day, e.g., about 1-10 g/day for an average person. In some embodiments, the daily dosage is from about 0.01 g to about 5 g, about 1 to about 10 g, about 5 g to about 20 g, about 10 g to about 50 g, about 20 g to about 100 g, or about 50 g to about 100 g.

In some instances, each administration of a therapeutic (e.g., an active agent) is in an amount of about: 0.01-1 g, 0.1-5 g, 0.1-10 g, 1-5 g, 1-10 g, 1-20 g, 10-20 g, 10-30 g, 10-40 g, 10-50 g, 20-30 g, 20-40 g, 20-50 g, 25-50 g, 30-40 g, 30-50 g, 30-60 g, 40-50 g, 40-60 g, 50-60 g, 50-75 g, 60-80 g, 75-100 g, or 80-100 g, for example: about 0.5 g, about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, about 10 g, about 10.5 g, about 11 g, about 11.5 g, about 12 g, about 12.5 g, about 13 g, about 13.5 g, about 14 g, about 14.5 g, about 15 g, about 15.5 g, about 16 g, about 16.5 g, about 17 g, about 17.5 g, about 18 g, about 18.5 g, about 19 g, about 19.5 g, about 20 g, about 22.5 g, about 25 g, about 27.5 g, about 30 g, about 32.5 g, about 35 g, about 37.5 g, about 40 g, about 42.5 g, about 45 g, about 47.5 g, about 50 g, about 55 g, about 60 g, about 65 g, about 70 g, about 75 g, about 80 g, about 85 g, about 90 g, about 95 g, or about 100 g.

In some instances, a therapeutic (e.g., in a liquid) administered to a subject having an active agent concentration of about: 0.01-0.1, 0.1-1, 1-10, 1-20, 5-30, 5-40, 5-50, 10-20, 10-25, 10-30, 10-40, 10-50, 15-20, 15-25, 15-30, 15-40, 15-50, 20-30, 20-40, 20-50, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 50-150, 50-200, 50-300, 100-300, 100-400, 100-500, 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, or 1000 μM, or any combination thereof.

In some cases, a therapeutic can comprise one or more active agents, administered to a subject at least about: 0.001 mg, 0.01 mg, 0.1 mg, 0.2 mg, 0.3 mg, 0.4 mg, 0.5 mg, 0.6 mg, 0.7 mg, 0.8 mg, 0.9 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg, or per kg body weight of a subject in need thereof. The therapeutic may comprise a total dose of one or more active agents administered at about 0.1 to about 10.0 mg, for example, about 0.1-10.0 mg, about 0.1-9.0 mg, about 0.1-8.0 mg, about 0.1-7.0 mg, about 0.1-6.0 mg, about 0.1-5.0 mg, about 0.1-4.0 mg, about 0.1-3.0 mg, about 0.1-2.0 mg, about 0.1-1.0 mg, about 0.1-0.5 mg, about 0.2-10.0 mg, about 0.2-9.0 mg, about 0.2-8.0 mg, about 0.2-7.0 mg, about 0.2-6.0 mg, about 0.2-5.0 mg, about 0.2-4.0 mg, about 0.2-3.0 mg, about 0.2-2.0 mg, about 0.2-1.0 mg, about 0.2-0.5 mg, about 0.5-10.0 mg, about 0.5-9.0 mg, about 0.5-8.0 mg, about 0.5-7.0 mg, about 0.5-6.0 mg, about 0.5-5.0 mg, about 0.5-4.0 mg, about 0.5-3.0 mg, about 0.5-2.0 mg, about 0.5-1.0 mg, about 1.0-10.0 mg, about 1.0-5.0 mg, about 1.0-4.0 mg, about 1.0-3.0 mg, about 1.0-2.0 mg, about 2.0-10.0 mg, about 2.0-9.0 mg, about 2.0-8.0 mg, about 2.0-7.0 mg, about 2.0-6.0 mg, about 2.0-5.0 mg, about 2.0-4.0 mg, about 2.0-3.0 mg, about 5.0-10.0 mg, about 5.0-9.0 mg, about 5.0-8.0 mg, about 5.0-7.0 mg, about 5.0-6.0 mg, about 6.0-10.0 mg, about 6.0-9.0 mg, about 6.0-8.0 mg, about 6.0-7.0 mg, about 7.0-10.0 mg, about 7.0-9.0 mg, about 7.0-8.0 mg, about 8.0-10.0 mg, about 8.0-9.0 mg, or about 9.0-10.0 mg, or per kg body weight of a subject in need thereof.

In some cases, a method of treatment disclosed herein comprises administering a therapeutic. In some instances, the method comprises administering a therapeutic includes one or more of the following steps: a) obtaining a genetic material sample of a human female subject, b) identifying in the genetic material of the subject a genetic marker having an association with endometriosis, c) assessing the subject's risk of endometriosis or risk of endometriosis progression, d) identifying the subject as having an altered risk of endometriosis or an altered risk of endometriosis progression, e) administering to the subject a therapeutic, or any combination thereof.

In some instances, the subject may be endometriosis presymptomatic or the subject may exhibit endometriosis symptoms. In some instances, the assessment of risk may include non-genetic clinical factors. In some instances, the therapeutic is adapted to the specific subject so as to be a proper and effective amount of therapeutic for the subject. In some instances, the administration of the therapeutic may comprise multiple sequential instances of administration of the therapeutic and that such sequence instances may occur over an extended period of time or may occur on an indefinite on-going basis. In some instances, the therapeutic may be a gene or protein based therapy adapted to the specific needs of a select patient.

Hormonal Therapy

In some cases, a treatment method herein comprises supplementing the body with a hormone thereof such as a steroid hormone, for example a method of preventing endometriosis comprising administering a hormonal therapy to a human subject having at least one genetic variant defining a minor allele disclosed herein, e.g., listed in Table 1 or 2. In some instances, the hormone can be progestin, progestogen, progesterone, desogestrel, etonogestrel, gestodene, levonorgestrel, medroxyprogesterone, norethisterone, norgestimate, megestrol, megestrol acetate, norgestrel, a pharmaceutically acceptable salt thereof (e.g., acetate), or any combination thereof. In some instances, a therapeutic used herein is selected from progestins, estrogens, antiestrogens, and antiprogestins, for example micronized danazol in a micro- or nanoparticulate formulation. Methods and therapeutics presented herein can utilize an active agent in a freebase, salt, hydrate, polymorph, isomer, diastereomer, prodrug, metabolite, ion pair complex, or chelate form. An active agent can be formed using a pharmaceutically acceptable non-toxic acid or base, including an inorganic acid or base, or an organic acid or base. In some instances, an active agent that can be utilized in connection with the methods and compositions presented herein is a pharmaceutically acceptable salt derived from acids including, but not limited to, the following: acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, or p-toluenesulfonic acid. For further description of pharmaceutically acceptable salts that can be used in the methods described herein see, for example, S. M. Barge et al., “Pharmaceutical Salts,” 1977, J. Pharm. Sci. 66:1-19, which is incorporated herein by reference in its entirety.

In some instances, the therapeutic may take the form of a testosterone or a modified testosterone such as Danazol. In some instances, the therapeutic can be a hormonal treatment therapeutic which may be administered alone or in combination with a gene therapy. For instance, the therapeutic may be an estrogen containing composition, a progesterone containing composition, a progestin containing composition, a gonadotropin releasing-hormone (GnRH) agonist, a gonadotropin releasing-hormone (GnRH) antagonist such as Elagolix, or other ovulation suppression composition, or a combination thereof. In some instances, the GnRH agonist may take the form of a GnRH agonist in combination with a patient specific substantially low dose of estrogen, progestin, or tibolone via an add-back administration. In some instances, in such add-back therapy, the dosage of estrogen, progestin, or tibolone is relatively small so as to not reduce the effectiveness of the GnRH agonist. In some instances, the therapeutic is an oral contraceptive (OC). In some instances, the OC is in a pill form that is comprised at least partially of estrogen, progesterone, or a combination thereof. In some instances, the progesterone component may be any of Desogestrel, Drospirenone, Ethynodiol, Levonorgestrel, Norethindrone, Norgestimate, and Norgestrel, and the estrogen component may further be any of Mestranol, Estradiol, and Ethinyl. In some instances, the OC may be any commercially available OC including ALESSE, APRI, ARANELLE, AVIANE, BREVICON, CAMILA, CESIA, CRYSELLE, CYCLESSA, DEMULEN, DESOGEN, ENPRESSE, ERRIN, ESTROSTEP, JOLIVETTE, JUNEL, KARIVA, LEENA, LESSINA, LEVLEN, LEVORA, LOESTRIN, LUTERA, MICROGESTIN, MICRONOR, MIRCETTE, MODICON, MONONESSA, NECON, NORA, NORDETTE, NORINYL, NOR-QD, NORTREL, OGESTREL, ORTHO-CEPT, ORTHO-CYCLEN, ORTHO-NOVUM, ORTHO-TRI-CYCLEN, OVCON, OVRAL, OVRETTE, PORTIA, PREVIFEM, RECLIPSEN, SOLIA, SPRINTEC, TRINESSA, TRI-NORINYL, TRIPHASIL, TRIVORA, VELIVET, YASMIN, AND ZOVIA (the preceding names are the registered trademarks of the respective providers).

Assisted Reproductive Therapy

In some cases, a method herein can comprise administering to a select subject assisted reproductive therapy (ART), for example a method of treating endometriosis associated infertility comprising administering ART to a select human subject having at least one genetic variant defining a minor allele disclosed herein, e.g., listed in Table 2. In some instances, ART can comprise in vitro fertilization (IVF), embryo transfer (ET), fertility medication, intracytoplasmic sperm injection (ICSI), cryopreservation, or any combination thereof. In some instances, ART can comprise surgically removing eggs from a woman's ovaries, combining them with sperm in the laboratory, and returning them to the woman's body or donating them to another woman.

In some instances, the in vitro fertilization (IVF) procedure can provide for a live birth event following the IVF procedure. In some instances, a method herein provides a probability of a live birth event occurring resulting from the first or subsequent in vitro fertilization cycle based at least in part on items of information from the female subjects.

In some instances, the IVF can comprise ovulation induction, utilizing fertility medication can comprise agents that stimulate the development of follicles in the ovary. Examples are gonadotropins and gonadotropin releasing hormone.

In some instances, IVF can comprise transvaginal ovum retrieval (OVR), which can be a process whereby a small needle is inserted through the back of the vagina and guided via ultrasound into the ovarian follicles to collect the fluid that contains the eggs.

In some instances, IVF can comprise embryo transfer, which can be the step in the process whereby one or several embryos are placed into the uterus of the female with the intent to establish a pregnancy.

In some instances, IVF can comprise assisted zona hatching (AZH), which can be performed shortly before the embryo is transferred to the uterus. A small opening can be made in the outer layer surrounding the egg in order to help the embryo hatch out and aid in the implantation process of the growing embryo.

In some instances, IVF can comprise artificial insemination, for example intrauterine insemination, intracervical insemination, intrauterine tuboperitoneal insemination, intratubal insemination, or any combination thereof.

In some instances, IVF can comprise intracytoplasmic sperm injection (ICSI), which can be beneficial in the case of male factor infertility where sperm counts are very low or failed fertilization occurred with previous IVF attempt(s). The ICSI procedure can involve a single sperm carefully injected into the center of an egg using a microneedle. With ICSI, only one sperm per egg is needed. Without ICSI, one may need between 50,000 and 100,000. In some embodiments, this method can be employed when donor sperm is used.

In some instances, IVF can comprise autologous endometrial coculture, which can be a possible treatment for patients who have failed previous IVF attempts or who have poor embryo quality. The patient's fertilized eggs can be placed on top of a layer of cells from the patient's own uterine lining, creating a more natural environment for embryo development.

In some instances, IVF can comprise zygote intrafallopian transfer (ZIFT), in which egg cells can be removed from the woman's ovaries and fertilized in the laboratory; the resulting zygote can be then placed into the fallopian tube.

In some instances, IVF can comprise cytoplasmic transfer, in which the contents of a fertile egg from a donor can be injected into the infertile egg of the patient along with the sperm.

In some instances, IVF can comprise egg donors, which are resources for women with no eggs due to surgery, chemotherapy, or genetic causes; or with poor egg quality, previously unsuccessful IVF cycles or advanced maternal age. In the egg donor process, eggs can be retrieved from a donor's ovaries, fertilized in the laboratory with the sperm from the recipient's partner, and the resulting healthy embryos can be returned to the recipient's uterus.

In some instances, IVF can comprise sperm donation, which may provide the source for the sperm used in IVF procedures where the male partner produces no sperm or has an inheritable disease, or where the woman being treated has no male partner.

In some instances, IVF can comprise preimplantation genetic diagnosis (PGD), which can involve the use of genetic screening mechanisms such as fluorescent in-situ hybridization (FISH) or comparative genomic hybridization (CGH) to help identify genetically abnormal embryos and improve healthy outcomes.

In some instances, IVF can comprise embryo splitting can be used for twinning to increase the number of available embryos.

In some instances, ART can comprise gamete intrafallopian transfer (GIFT), in which a mixture of sperm and eggs can be placed directly into a woman's fallopian tubes using laparoscopy following a transvaginal ovum retrieval.

In some instances, ART can comprise reproductive surgery, treating e.g. fallopian tube obstruction and vas deferens obstruction, or reversing a vasectomy by a reverse vasectomy. In surgical sperm retrieval (SSR) the reproductive urologist can obtain sperm from the vas deferens, epididymis or directly from the testis in a short outpatient procedure. By cryopreservation, eggs, sperm and reproductive tissue can be preserved for later IVF.

In some instances, a subject to treat can be a pre-in vitro fertilization (pre-IVF) procedure patient. In certain embodiments, the items of information relating to preselected patient variables for determining the probability of a live birth event for a pre-IVF procedure patient may include age, diminished ovarian reserve, 3 follicle stimulating hormone (FSH) level, body mass index, polycystic ovarian disease, season, unexplained female infertility, number of spontaneous miscarriages, year, other causes of female infertility, number of previous pregnancies, number of previous term deliveries, endometriosis, tubal disease, tubal ligation, male infertility, uterine fibroids, hydrosalpinx, and male infertility causes.

In some instances, a subject to treat can be a pre-surgical (pre-OR) procedure patient (pre-OR is also referred to herein as pre-oocyte retrieval). In certain embodiments, the items of information relating to preselected patient variables for determining the probability of a live birth event for a pre-OR procedure patient may include age, endometrial thickness, total number of oocytes, total amount of gonatropins administered, number of total motile sperm after wash, number of total motile sperm before wash, day 3 follicle stimulating hormone (FSH) level, body mass index, sperm collection, age of spouse, season number of spontaneous miscarriages, unexplained female infertility, number of previous term deliveries, year, number of previous pregnancies, other causes of female infertility, endometriosis, male infertility, tubal ligation, polycystic ovarian disease, tubal disease, sperm from donor, hydrosalpinx, uterine fibroids, and male infertility causes.

In some instances, a subject to treat can be a post-in vitro fertilization (post-IVF) procedure patient. In certain embodiments, the items of information relating to preselected patient variables for determining the probability of a live birth event for a post-IVF procedure patient may include blastocyst development rate, total number of embryos, total amount of gonatropins administered, endometrial thickness, flare protocol, average number of cells per embryo, type of catheter used, percentage of 8-cell embryos transferred, day 3 follicle stimulating hormone (FSH) level, body mass index, number of motile sperm before wash, number of motile sperm after wash, average grade of embryos, day of embryo transfer, season, number of spontaneous miscarriages, number of previous term deliveries, oral contraceptive pills, sperm collection, percent of unfertilized eggs, number of embryos arrested at 4-cell stage, compaction on day 3 after transfer, percent of normal fertilization, percent of abnormally fertilized eggs, percent of normal and mature oocytes, number of previous pregnancies, year, polycystic ovarian disease, unexplained female infertility, tubal disease, male infertility only, male infertility causes, endometriosis, other causes of female infertility, uterine fibroids, tubal ligation, sperm from donor, hydrosalpinx, performance of ICSI, or assisted hatching.

Pain Managing Medications

In some cases, a method disclosed herein can comprise administering a pain medication to a select subject, for example to a human subject having at least one genetic variant defining a minor allele listed in Table 1 or 2. In some instances, the pain medication comprises a nonsteroidal anti-inflammatory drug (NSAID), ibuprofen, naproxen, acetaminophen, an opioid, a cannabis-based therapeutic, or any combination thereof.

In some instances, the pain medication described herein can comprise an NSAID, for example amoxiprin, benorilate, choline magnesium salicylate, diflunisal, faislamine, methyl salicylate, magnesium salicylate, diclofenac, aceclofenac, acemetacin, bromfenac, etodolac, indometacin, nabumetone, sulindac, tolmetin, ibuprofen, carprofen, fenbuprofen, flubiprofen, ketaprofen, ketorolac, loxoprofen, naproxen, suprofen, mefenamic acid, meclofenamic acid, piroxicam, lomoxicam, meloxicam, tenoxicam, phenylbutazone, azapropazone, metamizole, oxyphenbutazone, or sulfinprazone, or a pharmaceutically acceptable salt thereof.

In some instances, the pain medication described herein can comprise an opioid analgesic, for example hydrocodone, oxycodone, morphine, diamorphine, codeine, pethidine, alfentanil, buprenorphine, butorphanol, dezocine, fentanyl, hydromorphone, levomethadyl acetate, levorphanol, meperidine, methadone, morphine sulfate, nalbuphine, oxymorphone, pentazocine, propoxyphene, remifentanil, sufentanil, or tramadol, or a pharmaceutically acceptable salt thereof.

In some instances, the pain medication described herein can comprise a cannabis-based therapeutic such as a cannabinoid for the treatment, reduction or prevention of pain. Exemplary cannabinoid for the treatment of pain include, without limitation, nabilone, dronabinol (THC), cannabidiol (CBD), cannabinol (CBN), cannabichromeme (CBC), cannabigerol (CBG), tetrahydrocannabivarin (THCV), tetrahydrocannabinolic acid (THCA), cannabidivarin (CBDV), cannadidiolic acid (CBDA), ajulemic acid, dexanabinol, cannabinor, HU 308, HU 331, and a pharmaceutically acceptable salt thereof.

In some cases, the method comprises detecting in a genetic sample at least one genetic mutation in at least one gene of: UGT2B28, USP17L2, METTL11B, or any combination thereof. In some cases, the method comprises detecting in a genetic sample at least one genetic mutation in at least one gene of: UGT2B7, UGT2B11, UGT2B28, UGT2B4, CTSB, DEFB136, USP17L2, LONRF1, KIAA1456, METTL11B, or any combination thereof. In some cases, the method comprises detecting in a genetic sample at least one genetic mutation in at least one gene of: UGT2B11, UGT2B28, UGT2B4, CTSB, DEFB136, USP17L2, LONRF1, KIAA1456, METTL11B, or any combination thereof. In some cases, the method comprises detecting in a genetic sample at least one genetic mutation in at least one gene of: UGT2B7, UGT2B28, UGT2B4, CTSB, DEFB136, USP17L2, LONRF1, KIAA1456, METTL11B, or any combination thereof. In some cases, the method comprises detecting in a genetic sample at least one genetic mutation in at least one gene of: UGT2B7, UGT2B11, UGT2B4, CTSB, DEFB136, USP17L2, LONRF1, KIAA1456, METTL11B, or any combination thereof. In some cases, the method comprises detecting in a genetic sample at least one genetic mutation in at least one gene of: UGT2B7, UGT2B11, UGT2B28, CTSB, DEFB136, USP17L2, LONRF1, KIAA1456, METTL11B, or any combination thereof. In some cases, the method comprises detecting in a genetic sample at least one genetic mutation in at least one gene of: UGT2B7, UGT2B11, UGT2B28, UGT2B4, DEFB136, USP17L2, LONRF1, KIAA1456, METTL11B, or any combination thereof. In some cases, the method comprises detecting in a genetic sample at least one genetic mutation in at least one gene of: UGT2B7, UGT2B11, UGT2B28, UGT2B4, CTSB, USP17L2, LONRF1, KIAA1456, METTL11B, or any combination thereof. In some cases, the method comprises detecting in a genetic sample at least one genetic mutation in at least one gene of: UGT2B7, UGT2B11, UGT2B28, UGT2B4, CTSB, DEFB136, LONRF1, KIAA1456, METTL11B, or any combination thereof. In some cases, the method comprises detecting in a genetic sample at least one genetic mutation in at least one gene of: UGT2B7, UGT2B11, UGT2B28, UGT2B4, CTSB, DEFB136, USP17L2, KIAA1456, METTL11B, or any combination thereof. In some cases, the method comprises detecting in a genetic sample at least one genetic mutation in at least one gene of: UGT2B7, UGT2B11, UGT2B28, UGT2B4, CTSB, DEFB136, USP17L2, LONRF1, METTL11B, or any combination thereof. In some cases, the method comprises detecting in a genetic sample at least one genetic mutation in at least one gene of: UGT2B7, UGT2B11, UGT2B28, UGT2B4, CTSB, DEFB136, USP17L2, LONRF1, KIAA1456, or any combination thereof.

In some cases, the method comprises detecting in a genetic sample at least one genetic mutation in at least one gene of: UGT2B7, UGT2B28, USP17L2, METTL11B, or any combination thereof. In some cases, the method comprises detecting in a genetic sample at least one genetic mutation in at least one gene of: UGT2B11, UGT2B28, USP17L2, METTL11B, or any combination thereof. In some cases, the method comprises detecting in a genetic sample at least one genetic mutation in at least one gene of: UGT2B28, UGT2B4, USP17L2, METTL11B, or any combination thereof. In some cases, the method comprises detecting in a genetic sample at least one genetic mutation in at least one gene of: UGT2B28, CTSB, USP17L2, METTL11B, or any combination thereof. In some cases, the method comprises detecting in a genetic sample at least one genetic mutation in at least one gene of: UGT2B28, DEFB136, USP17L2, METTL11B, or any combination thereof. In some cases, the method comprises detecting in a genetic sample at least one genetic mutation in at least one gene of: UGT2B28, USP17L2, LONRF1, METTL11B, or any combination thereof. In some cases, the method comprises detecting in a genetic sample at least one genetic mutation in at least one gene of: UGT2B28, USP17L2, KIAA1456, METTL11B, or any combination thereof.

### SPECIFIC EMBODIMENTS

A number of methods and systems are disclosed herein. Specific exemplary embodiments of these methods and systems are disclosed below.

### Embodiment 1

A method comprising assaying a genetic sample of a patient, detecting in said sample at least one genetic mutation in at least one gene of UGT2B28, USP17L2, and METTL11B, and applying at least one endometriosis therapeutic to said patient.

### Embodiment 2

The method of embodiment 1, wherein said assaying comprises at least one of sequencing, array comparative genomic hybridization (CGH), polymerase chain reaction (PCR), or the use of a DNA microarray.

### Embodiment 3

The method of embodiment 1 or 2, wherein said at least one genetic mutation comprises at least one of a hemizygous deletion mutation and a rare missense mutation.

### Embodiment 4

The method of any one of embodiments 1-3, wherein said at least one genetic mutation comprises at least one of a hemizygous deletion mutation in at least one of UGT2B28 and USP17L2 and a rare missense mutation in METTL11B.

### Embodiment 5

The method of any one of embodiments 1-4, wherein said patient manifests at least one of pelvic pain, infertility, and dysmenorrhea.

### Embodiment 6

The method of any one of embodiments 1-5, wherein said endometriosis therapeutic comprises administering a hormonal treatment to said patient, canceling a contemplated hormonal treatment of said patient, performing a surgical procedure on said subject, or canceling a contemplated surgical procedure of said patient.

### Embodiment 7

The method of any one of embodiments 1-6, wherein said hormonal treatment comprises at least one of an estrogen containing composition, a progesterone containing composition, a progestin containing composition, a gonadotropin releasing-hormone (GnRH) agonist, a GnRH antagonist, and any combination thereof.

### Embodiment 8

A method comprising applying at least one endometriosis therapeutic to a patient having at least one genetic mutation in at least one gene of UGT2B28, USP17L2, and METTL11B in the DNA of said patient.

### Embodiment 9

The method of embodiment 8, wherein said at least one genetic mutation comprises at least one of a hemizygous deletion mutation and a rare missense mutation.

### Embodiment 10

The method of embodiment 8 or 9, wherein said at least one genetic mutation comprises at least one of a hemizygous deletion mutation in at least one of UGT2B28 and USP17L2 and a rare missense mutation in METTL11B.

### Embodiment 11

The method of any one of embodiments 8-10, wherein said patient manifests at least one of pelvic pain, infertility, and dysmenorrhea.

### Embodiment 12

The method of any one of embodiments 8-11, wherein said endometriosis therapeutic comprises administering a hormonal treatment to said patient, canceling a contemplated hormonal treatment of said patient, performing a surgical procedure on said subject, or canceling a contemplated surgical procedure of said patient

### Embodiment 13

The method of any one of embodiments 8-12, wherein said hormonal treatment comprises at least one of an estrogen containing composition, a progesterone containing composition, a progestin containing composition, a gonadotropin releasing-hormone (GnRH) agonist, and any combination thereof.

### Embodiment 14

The method of embodiment 1, wherein the genetic sample is obtained from a blood sample.

### Embodiment 15

The method of embodiment 1, further comprising a treatment for the subject, wherein the treatment comprises a recommendation for the treatment.

### Embodiment 16

The method of embodiment 1, wherein the detecting comprises comparing a data set obtained from the genetic sample to a control data set of a control sample.

### Embodiment 17

The method of embodiment 16, wherein the data set comprises sequencing data.

### Embodiment 18

The method of embodiment 16, wherein a portion of data from the data set is removed.

### Embodiment 19

The method of embodiment 16, wherein a portion of data from the control data set is removed.

### Embodiment 20

The method of embodiment 18 or embodiment 19, wherein an accuracy of the detecting is improved after a removal of the portion of data.

### Embodiment 21

The method of embodiment 18 or embodiment 19, wherein a false positive rate of the detecting is reduced after a removal of the portion of data.

### Embodiment 22

The method of embodiment 19, wherein the portion of data removed from the control data set is data of a sample that is familial to the genetic material.

### Embodiment 23

The method of embodiment 16, wherein the control sample is selected based on one or more parameters of associated with the genetic material.

### Embodiment 24

The method of embodiment 23, wherein the one or more parameters comprise an ethnicity, an age, a gender, a geographical location, a diet, a medical history, a familial history, a sample preparation, or any combination thereof.

### Embodiment 25

A method comprising: (a) hybridizing a nucleic acid probe to a nucleic acid sample from a human subject suspected of having or developing endometriosis; and (b) detecting a genetic variant in a panel comprising two or more genetic variants defining a minor allele listed in Tables 1 and 2.

### Embodiment 26

The method of embodiment 25, wherein the nucleic acid sample comprises mRNA, cDNA, genomic DNA, or PCR amplified products produced therefrom, or any combination thereof.

### Embodiment 27

The method of embodiment 25 or 26, wherein the nucleic acid sample comprises PCR amplified nucleic acids produced from cDNA or mRNA.

### Embodiment 28

The method of embodiment 25 or 26, wherein the nucleic acid sample comprises PCR amplified nucleic acids produced from genomic DNA.

### Embodiment 29

The method of any one of embodiments 25-28, wherein the nucleic acid probe is a sequencing primer.

### Embodiment 30

The method of any one of embodiments 25-28, wherein the nucleic acid probe is an allele specific probe.

### Embodiment 31

The method of any one of embodiments 25-30, wherein the detecting comprises DNA sequencing, hybridization with a complementary probe, an oligonucleotide ligation assay, a PCR-based assay, or any combination thereof.

### Embodiment 32

The method of embodiment 25, wherein the detecting yields a data set.

### Embodiment 33

The method of embodiment 31, further comprising inputting the data set into a programmed computer having a trained algorithm.

### Embodiment 34

The method of embodiment 32, further comprising outputting an electronic report that comprises a result.

### Embodiment 35

The method of embodiment 25, wherein the detecting comprises sequencing and wherein the sequencing comprises next-gen sequencing.

### Embodiment 36

The method of embodiment 25, wherein the detecting comprises sequencing and wherein the sequencing comprises nanopore sequencing.

### Embodiment 37

The method of embodiment 35, wherein the nanopore sequencing is performed with a biological nanopore, a solid state nanopore, a hybrid nanopore, or any combination thereof.

### Embodiment 38

The method of embodiment 25, wherein the detecting comprises labeling the one or more SNPs.

### Embodiment 39

The method of embodiment 37, wherein the labeling comprises associating a fluorescent label with the one or more SNPs.

### Embodiment 40

The method of embodiment 37, wherein the labeling comprises covalently labeling the one or more SNPs.

### Embodiment 41

The method of embodiment 25, wherein the nucleic acid sample is at least partially isolated from a blood sample.

### Embodiment 42

The method of embodiment 25, wherein the nucleic acid sample is at least partially isolated from a cell-free sample.

### Embodiment 43

The method of embodiment 25, wherein the nucleic acid sample is comprised in a cell-free DNA.

### Embodiment 44

The method of any one of embodiments 25-43, wherein the panel comprises at least: 5, 10, 15, or 20 genetic variants defining minor alleles listed in Tables 1 and 2.

### Embodiment 45

The method of any one of embodiments 25-44, wherein the genetic variant comprises a synonymous mutation, a non-synonymous mutation, a nonsense mutation, an insertion, a deletion, a splice-site variant, a frameshift mutation, or any combination thereof.

### Embodiment 46

The method of any one of embodiments 25-45, wherein the genetic variant comprises a protein damaging mutation.

### Embodiment 47

The method of any one of embodiments 25-46, wherein the panel comprises one or more protein damaging or loss of function variants in one or more genes selected from the group consisting of UGT2B28, USP17L2, METTL11B, and any combinations thereof.

### Embodiment 48

The method of any one of embodiments 25-47, further comprising sequencing the one or more genes to identify one or more protein damaging or loss of function variants.

### Embodiment 49

The method of embodiment 48, wherein the one or more protein damaging or loss of function variants is identified based on a predictive computer algorithm.

### Embodiment 50

The method of embodiment 48, wherein the one or more protein damaging or loss of function variants is identified based on reference to a database.

### Embodiment 51

The method of embodiment 47, wherein the one or more protein damaging or loss of function variants comprises a stop-gain mutation, a splice-site mutation, a frameshift mutation, a missense mutation, or any combination thereof.

### Embodiment 52

The method of e any one of embodiments 25-51, wherein the panel is capable of identifying a human subject as having or being at risk of developing endometriosis with a specificity of at least: 80%, 85%, 90%, 95%, 96%, 97%, 98%, or 99%.

### Embodiment 53

The method of any one of embodiments 25-52, wherein the panel is capable of identifying a human subject as having or being at risk of developing endometriosis with a sensitivity of at least: 80%, 85%, 90%, 95%, 96%, 97%, 98%, or 99%.

### Embodiment 54

The method of any one of embodiments 25-53, wherein the panel is capable of identifying a human subject as having or being at risk of developing endometriosis with an accuracy of at least: 80%, 85%, 90%, 95%, 96%, 97%, 98%, or 99%.

### Embodiment 55

The method of any one of embodiments 25-54, further comprising administering a therapeutic to the human subject.

### Embodiment 56

The method of embodiment 55, wherein the therapeutic comprises a regenerative therapy, a medical device, a pharmaceutical composition, a medical procedure, or any combination thereof.

### Embodiment 57

The method of embodiment 56, wherein the therapeutic comprises a non-steroidal anti-inflammatory, a hormone treatment, a dietary supplement, a cannabis-derived therapeutic or any combination thereof.

### Embodiment 58

The method of embodiment 56, wherein the therapeutic comprises the pharmaceutical composition, and wherein the pharmaceutical composition comprises an at least partially hemp-derived therapeutic, an at least partially cannabis-derived therapeutic, a cannabidiol (CBD) oil derived therapeutic, or any combination thereof.

### Embodiment 59

The method of embodiment 56, wherein the therapeutic comprises the medical procedure, and wherein the medical procedure comprises a laparoscopy, a laser ablation procedure, a hysterectomy, or any combination thereof.

### Embodiment 60

The method of embodiment 56, wherein the therapeutic comprises the regenerative therapy, and wherein the regenerative therapy comprises a stem cell, a cord blood cell, a Wharton's jelly, an umbilical cord tissue, a tissue, or any combination thereof.

### Embodiment 61

The method of embodiment 56, wherein the therapeutic comprises the pharmaceutical composition, and wherein the pharmaceutical composition comprises cannabis, cannabidiol oil, hemp, or any combination thereof.

### Embodiment 62

The method of embodiment 56, wherein the therapeutic comprises the pharmaceutical composition, and wherein the pharmaceutical composition is formulated in a unit dose.

### Embodiment 63

The method of embodiment 55, wherein the therapeutic comprises hormonal therapy, an advanced reproductive therapy, a pain managing medication, or any combination thereof.

### Embodiment 64

The method of embodiment 55, wherein the therapeutic comprises a hormonal contraceptive, gonadotropin-releasing hormone (GnRH) agonist, gonadotropin-releasing hormone (GnRH) antagonist, progestin, danazol, or any combination thereof.

### Embodiment 65

The method of any one of embodiments 25-64, further comprising administering an imaging procedure to a subject.

### Embodiment 66

The method of embodiment 65, wherein the imaging procedure comprises an ultrasound, an x-ray, a magnetic resonance imaging (MRI), a computed tomography (CT) scan, or any combination thereof.

### Embodiment 67

The method of any one of embodiments 25-66, wherein the human subject is asymptomatic for endometriosis.

### Embodiment 68

The method of any one of embodiments 25-67, wherein the human subject is a teenager.

### Embodiment 69

A method comprising detecting one or more genetic variants defining a minor allele listed in Tables 1 and 2 in genetic material from a human subject suspected of having or developing endometriosis.

### Embodiment 70

The method of embodiment 69, wherein the genetic material comprises mRNA, cDNA, genomic DNA, or PCR amplified products produced therefrom, or any combination thereof.

### Embodiment 71

The method of embodiment 69 or 70, wherein the detecting comprises DNA sequencing, hybridization with a complementary probe, an oligonucleotide ligation assay, a PCR-based assay, of any combination thereof.

### Embodiment 72

The method of any one of embodiments 69-71, wherein the detecting comprises hybridizing a nucleic acid probe to the genetic material.

### Embodiment 73

The method of any one of embodiments 69-72, wherein the detecting comprises testing for the presence or absence of at least: 2, 3, 4, 5, 6, 7, 8, 9, 10, 15, or 20 genetic variants defining a minor allele listed in Table 1 and Table 2.

### Embodiment 74

The method of any one of embodiments 69-73, further comprising administering a therapeutic to the human subject.

### Embodiment 75

A method comprising: (a) sequencing all or a portion of one or more genes or gene expression products selected from the group consisting of UGT2B28, USP17L2, METTL11B and any combinations thereof to identify one or more protein damaging or loss of function variants in a human subject suspected of having or developing endometriosis; and (b) diagnosing the human subject as having or being at risk of developing when one or more protein damaging or loss of function variant is identified.

### Embodiment 76

The method of embodiment 75, wherein the one or more protein damaging or loss of function variants comprises a deletion of all or a portion of the one or more genes.

### Embodiment 77

The method of embodiment 75 or 76, wherein the one or more protein damaging or loss of function variants is identified based on a predictive computer algorithm, reference to a database, or a combination thereof.

### Embodiment 78

The method of any one of embodiments 75-77, wherein the one or more protein damaging or loss of function variants comprises a stop-gain mutation, a splice-site mutation, a frameshift mutation, a missense mutation, or any combination thereof.

### Embodiment 79

The method of any one of embodiments 75-78, further comprising administering a hormonal therapy to the human subject.

### Embodiment 80

The method of embodiment 79, wherein the hormonal therapy comprises administration of hormonal contraceptives, gonadotropin-releasing hormone (GnRH) agonists, gonadotropin-releasing hormone (GnRH) antagonists, progestin, danazol, or any combination thereof.

### Embodiment 81

The method of any one of embodiments 75-80, further comprising administering to the human subject an assisted reproductive therapy.

### Embodiment 82

The method of embodiment 81, wherein the assisted reproductive therapy comprises in vitro fertilization, intrauterine insemination, ovulation induction, gamete intrafallopian transfer, or any combination thereof.

### Embodiment 83

The method of any one of embodiments 75-82, further comprising administering to the human subject a pain medication.

### Embodiment 84

The method of embodiment 83, wherein the pain medication comprises a nonsteroidal anti-inflammatory drug (NSAID), ibuprofen, naproxen, an opioid, a cannabis-based therapeutic, or any combination thereof.

### Embodiment 85

The method of any one of embodiments 75-84, further comprising detecting the at least one genetic variant in a genetic material from the human subject.

### Embodiment 86

The method of embodiment 85, wherein the detecting comprises DNA sequencing, hybridization with a complementary probe, an oligonucleotide ligation assay, a PCR-based assay, or any combination thereof.

### Embodiment 87

The method of embodiment 85 or 86, wherein the detecting comprises hybridizing a nucleic acid probe to the genetic material.

### Embodiment 88

The method of embodiment 87, wherein the nucleic acid probe is a sequencing primer or an allele-specific probe.

### Embodiment 89

The method of any one of embodiments 75-88, wherein the human subject has at least one genetic variant that comprises a synonymous mutation, a non-synonymous mutation, a nonsense mutation, an insertion, a deletion, a splice-site variant, a frameshift mutation, or any combination thereof.

### Embodiment 90

The method of any one of embodiments 1-89, wherein the genetic variant has an odds ratio (OR) of at least about: 1, 1.5, 2, 5, 10, 20, 50, 100, or greater.

### Embodiment 91

A kit comprising: one or more probes for detecting one or more single nucleotide polymporphisms (SNPs) of Table 1, Table 2, or a combination thereof in a sample.

### Embodiment 92

The kit of embodiment 91, further comprising a control sample.

### Embodiment 93

The kit of embodiment 91, wherein the control sample comprises one or more of SNPs of Table 1, Table 2, or a combination thereof.

### Embodiment 94

The kit of embodiment 91, wherein a probe of the one or more probes comprises a sequence having at least 80% sequence complementarity to a sequence adjacent thereto a SNP of the one or more SNPs of Table 1, Table 2, or a combination thereof.

### Embodiment 95

The kit of embodiment 91, wherein the one or more probes comprise a hybridization probe or amplification primer.

### Embodiment 96

The kit of embodiment 91, wherein the one or more probes is configured to detect a variant allele in the sample.

### Embodiment 97

The kit of embodiment 91, wherein the one or more probes is configured to hybridize to a portion of a nucleic acid of the sample when a variant allele is present in the nucleic acid.

### Embodiment 98

The kit of embodiment 91, wherein the one or more probes is configured to associate with a solid support.

### Embodiment 99

The kit of embodiment 91, wherein the kit further comprises instructions for use and wherein the instructions for use comprise high stringent hybridization conditions.

### Embodiment 100

The kit of embodiment 91, wherein the one or more probes is configured to hybridize to a target region of a nucleic acid of the sample, wherein the target region comprises one or more SNPs.

### Embodiment 101

A system comprising: (a) a computer processor configured to receive sequencing data obtained from assaying a sample, wherein the computer processor is configured to identify a presence or an absence of one or more SNPs comprising one or more SNPs of Table 1, Table 2, or a combination thereof in the sample, and (b) a graphical user interface configured to display a report comprising the identification of the presence or the absence of the one or more SNPs in the sample.

### Embodiment 102

The system of embodiment 101, wherein the computer processor comprises a trained algorithm.

### Embodiment 103

The system of embodiment 101, wherein the computer processor communicates a result.

### Embodiment 104

The system of embodiment 103, wherein the result comprises an identification of the presence or the absence of one or more SNPs in the sample.

### EXAMPLES

### Example 1. Whole Exome Sequencing in a Greek Family Identifies Inherited Variations in Endometriosis

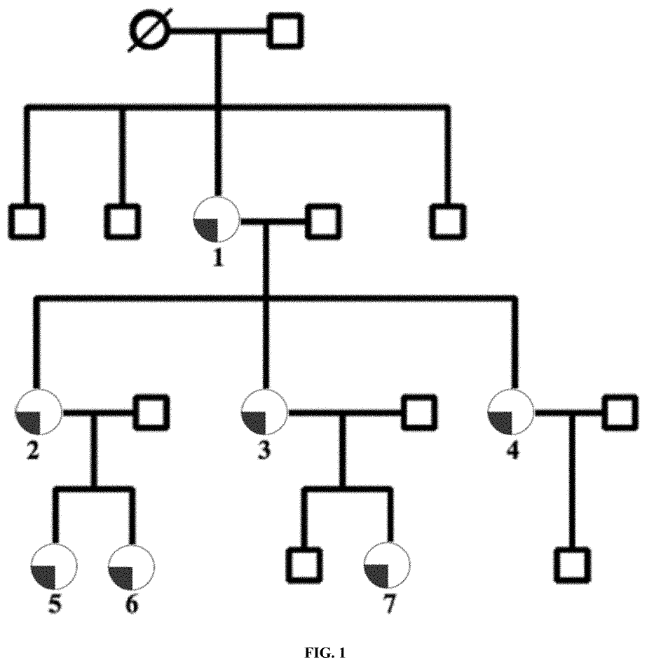

The pedigree of the studied Greek family of three generations is shown in FIG. 1 (also see Matalliotakis et al. Mol. Med. Rep. 2017 November; 16(5):6077-6080). Case no. 1 is a 65 years old female with severe endometriosis (stage IV, pelvic pain dysmenorhea symptoms), who suffered TAH (total abdominal hysterectomy) at age 32. She gave birth to three daughters with endometriosis (case nos. 2, 3 and 4) of a varying severity. The first daughter (no. 2) was 49 years old at the study, had severe endometriosis (stage IV, pelvic pain dysmenorrhea symptoms) and TAH at age 33, and gave birth to two daughters (case nos. 5 and 6). The second daughter (no. 3) was 46 years old at the study, had mild endometriosis (stage II, pelvic pain) and endometrioma, and gave birth to one daughter (case no. 7). The third daughter (no. 4) was 40 years old at the study, had endometriosis (stage II, infertility dysmenorhea) and adenomyosis, and gave birth to one son. The first granddaughter (case no. 5) was 32 years old at the study with endometriosis (stage III, infertility) and endometrioma, and had 2 children via in vitro fertilization (IVF). The second granddaughter (case no. 6) was 27 years old at the study with endometriosis (stage II, infertility pelvic pain) and endometrioma, and had no children. The third granddaughter (case no. 7) was 25 years old at the study with endometriosis (stage II, infertility) and endometrioma, and had no children.

Results: Hemizygous deletions in UGT2B28 and USP17L2 (alias DUBS) are associated with endometriosis in a Greek family, see Table 1 below. Rare missense mutations in METTL11B are associated with endometriosis in the same Greek family.

Table 1 shows two genomic regions around UGT2B28 and USP17L2 where inherited deletions have been identified. The positions identified with the italics correspond to the hemizygous deletions identified in the grandmother (Greece 1) and several of her decedents. The fields in the table with ./. are interpreted as wild-type homozygous; fields with 0/1 as heterozygotes, and 1/1 as homozygous for the alternate allele. Inheritance analysis reveals inconsistencies that is only compatible with a hemizygous deletion in the grandmother in each of the regions identified by italics.

### Example 2. Whole Exome Sequencing of High Risk Endometriosis Families

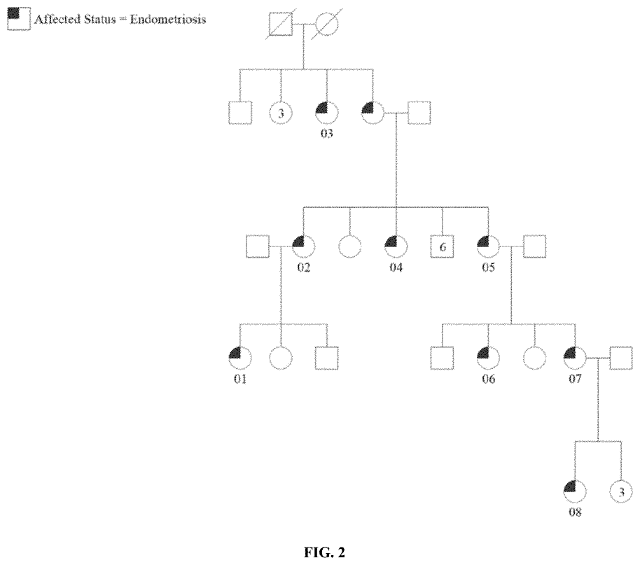

Two high risk endometriosis families were sequenced using Ampliseq Sequencing on Ion Proton. ESP_148 family (pedigree shown in FIG. 2) has 8 affected women, while the Greek family (pedigree shown in FIG. 1) has 7 affected women. Whole exome sequencing of affected individuals with 100×-fold mean coverage; 90% of genes had coverage at 20× or better. Coding variants with publicly available gnomAD dataset (non-finnish European Ancestry) of MAF (minor allele frequency)<1% were considered. Variants were further filtered to pathogenic damaging mutations (in-silico) shared by 5 or more affected women in each family. A shown in Table 2 below, one gene METTL11B was identified and shares a low frequency damaging missense mutation (p.L277P) in 5 affected women in Greek family and a distinct low frequency damaging missense mutation (p.M66T) in the ESP_148 family shared by 5 affected women. A low frequency damaging missense mutation (p.D18H) was identified present in one additional affected member in the Greek family.

### Example 3

A cell-free sample will be obtained from a human subject at risk of developing endometriosis. Next generation sequencing will be performed on the cell-free sample to detect a presence or an absence of one or more SNPs of Table 1, Table 2, or a combination thereof. A report will be generated with a classification of the cell-free sample based on the detected presence or absence of the one or more SNPs of Table 1, Table 2, or combination thereof. The classification will confirm whether the subject is at risk of developing endometriosis.

### Example 4

A blood sample will be obtained from a canine subject symptomatic for endometriosis. Nanopore sequencing will be performed on a portion of the sample to detect one or more SNPs of Table 1, Table 2, or a combination thereof. Results of the nanopore sequencing will be input into a trained algorithm. An output from the trained algorithm will identify a stage of endometriosis of the canine subject.

### Example 5

A subject will complete a medical questionnaire A subject will provide a sample for sequencing analysis. A presence or absence of one or more SNPs of Table 1, Table 2, or a combination thereof will be detected in the sample. Results of the medical questionnaire and the sequencing analysis will provide a stratified classification of the subject having either a low risk or high risk of developing endometriosis.

### Example 6

A subject asymptomatic for endometriosis will provide a sample as part of a screening exam. The sample will be analyzed for a presence or an absence of one or more SNPs of Table 1, Table 2, or any combination thereof. The results of the analysis will be compared to a reference. Based on a comparison to the result, a subject will receive an indication of risk of developing endometriosis in the future.

### Example 7

A sample obtained from a subject suspected of having endometriosis will be assayed for a plurality of SNPs including UGT2B28, USP17L2, METTL11B, or any combination thereof. A result of the assaying will be input into a trained algorithm. The trained algorithm will output a result including a classification of a presence or an absence of endometriosis in the sample at an accuracy of at least about 85%.

### Example 8

A sample will be assayed using a plurality of primers. One or more primers of the plurality of primers will comprise about 85% sequence complementarity to at least a portion of UGT2B28, USP17L2, METTL11B, or any combination thereof. The assaying will identify a presence or an absence of one or more SNPs in the sample.

### Example 9

A trained algorithm will be trained with a training set of samples. The training set of samples will comprise samples obtained from at least one subject confirmed to have endometriosis. The trained algorithm will utilize feature selection to rank or weight a plurality of SNPs. The ranking or weighting will identify SNPs of the plurality of SNPs to include in a biomarker panel to improve an accuracy of a result (including presence or absence of endometriosis in a sample) obtained by the trained algorithm.

### Example 10

An independent sample, separate from a training set of samples, will be obtained from a subject in need thereof and will be assayed for a presence of a plurality of SNPs, including a biomarker panel identified using the training set of samples. The biomarker panel will include UGT2B28, USP17L2, METTL11B, or any combination thereof. A result obtained from the assaying will be input to the trained algorithm. The trained algorithm will identify a presence or an absence of endometriosis in the independent sample with an accuracy of at least 85%.

### Example 11

Samples were run on a next generation sequencing platform, specifically on an Ion Proton system. Whole Exome sequencing (WES) was performed using Ampliseq sequencing. Samples run on WES were then aligned using a Texas Medication Algorithm Project (TMAP) algorithm and variants were called using a Torrent Variant caller with the default parameter settings as established by the manufacturer.

Samples that fell below the two standard deviation from average counts of the coding variant were eliminated from further analysis due to poor sequencing quality. Those samples eliminated from further analysis, if not removed, may contribute to spurious association results.

Population-based association analysis was performed on samples Familial samples, if included in the case population, may bias association results. Therefore, Identity By Descent (IBD) analysis was performed to remove any samples that were closely related (pi_hat <0.2).

Variants were annotated to distinguish the type of protein change (i.e synonymous, missense, splicing, stop gain, stop loss, frameshift etc).

Variants may differ significantly across different ethnic groups and thereby influence association results. Hence, it may be paramount to compare the case population (of a particular ethnic composition) against a control group having a similar ethnic composition, such as a reference population. Principal Component Analysis (PCA) was performed to assign various samples of the case population to distinct ethnic groups. In this study, Caucasian or Northern European ancestry was selected as the ethnic group. Association was performed using Caucasian subjects having endometriosis against a Non-Finnish European cohort obtained from a gnomad database. Samples of the gnomad database were primarily run on an Illumina sequencing platform across different laboratories. In order to eliminate association results potentially influenced by sequencing platform artifacts, the associated results were verified against Caucasian control subjects run using an Ion Proton system.

Homopolymer regions surrounding the variant of interest as well as variants called primarily on unidirectional sequencing strands may also add spurious association. Therefore, associated results were further subjected to visual verification. Visual verification may require each individual variant verified using the bam file on sequence visualization software.

A sample may be compared to a control or reference sample or one or more samples obtained from a reference population. Sequencing data obtained from a sample may be compared to sequencing data obtained from a control or reference sample. A data set obtained from a sample may be compared to a data set obtained from a control or reference sample. A control or reference sample may be selected based on one or more parameters associated with the sample (such as an ethnicity, age, gender, geographical location, diet, medical history, familial history, or others).

Confounding effects may be removed from a data set obtained from a sample, such as sequencing data set. Removal of confounding effects may improve a diagnostic accuracy, sensitivity, specificity, or any combination thereof of a method as described herein. For example, samples having less than about: 5, 4, 3, 2.5, 2, 1.5, 1 standard deviation from average counts of a coding variant may be removed from a data set. Data obtained from samples identified as familial samples relative to the sample of interest may be removed from a data set. A data set may be compared to a reference or control data set having similar ethnicity. Data obtained from homopolymer regions surrounding a variant of interest may be removed from a data set. Data obtained for variants called primarily on unidirectional sequencing strands may be removed from a data set. Any of the forgoing alone or in combination may be confounding effects that may be removed from a data set to yield an improved diagnostic accuracy, sensitivity, specificity, or combination thereof of a method as described herein.

Confounding effects may be removed from a data set prior to a comparison to a control or reference sample Confounding effects may be removed after a comparison. Samples identified as familial samples may be removed prior to obtaining a data set, such as prior to sequencing.

TABLE 1
Variants associated with endometriosis

| | | REF | Minor | | | Nucle | AA | |
|---|---|---|---|---|---|---|---|---|
| Chromosome | Position | Allele | Allele | Gene | Type | change | change | dbsnp |
| 4 | 69,964,337 | AT | TC | UGT2B7 | nonframeshift | c.801_802TC | | rs386675647 |
| | | | | | sub | | | |
| 4 | 69,972,949 | C | G | UGT2B7 | synonymous | c.C1059G | p.L353L | rs4292394 |
| 4 | 70,079,838 | T | C | UGT2B11 | synonymous | c.A603G | p.L201L | rs4694697 |
| 4 | 70,079,963 | G | A | UGT2B11 | synonymous | c.C478T | p.L160L | rs72551399 |
| 4 | 70,146,230 | G | A | UGT2B28 | synonymous | c.G12A | p.K4K | rs13139691 |
| 4 | 70,146,704 | G | A | UGT2B28 | synonymous | c.G486A | p.A162A | rs7689398 |
| 4 | 70,146,804 | G | C | UGT2B28 | nonsynonymous | c.G586C | p.V196L | rs148987832 |
| 4 | 70,156,302 | A | T | | missing | | | |
| 4 | 70,156,392 | A | G | UGT2B28 | synonymous | c.A1173G | p.V391V | rs10013145 |
| 4 | 70,160,277 | T | G | UGT2B28 | nonsynonymous | c.T1340G | p.I447R | rs6843900 |
| 4 | 70,160,309 | C | G | UGT2B28 | nonsynonymous | c.C1372G | p.H458D | rs6828191 |
| 4 | 70,160,338 | G | C | UGT2B28 | synonymous | c.G1401C | P.V467V | rs72552703 |
| 4 | 70,160,342 | TG | CC | UGT2B28 | nonframeshift | c.1405_1406CC | | rs796618077 |
| | | | | | sub | | | |
| 4 | 70,346,564 | GA | TT | UGT2B4 | nonframeshift | c.1374_1375AA | | rs67904882 |
| | | | | | sub | | | |
| 4 | 70,355,211 | T | C | UGT2B4 | synonymous | c.A948G | p.T316T | rs1845555 |
| 8 | 11,706,581 | T | G | CTSB | synonymous | c.A420C | p.T140T | rs13332 |
| 8 | 11,710,888 | G | C | CTSB | nonsynonymous | c.C76G | p.L26V | rs12338 |
| 8 | 11,832,079 | A | G | DEFB136 | synonymous | c.T30C | p.F10F | rs10108075 |
| 8 | 11,994,716 | C | A | USP17L2 | nonsynonymous | c.G1554T | p.K518N | rs199935289 |
| 8 | 11,994,957 | T | C | USP17L2 | nonsynonymous | c.A1313G | p.K438R | rs12543578 |
| 8 | 11,995,062 | G | A | USP17L2 | nonsynonymous | c.C1208T | p.A403V | rs75807755 |
| 8 | 12,600,720 | T | A | LONRF1 | nonsynonymous | c.A793T | p.I265L | rs1139354 |
| 8 | 12,878,637 | A | G | KIAA1456 | nonsynonymous | c.A449G | p.H150R | rs528255 |
| 8 | 12,878,677 | T | C | KIAA1456 | synonymous | c.T489C | p.A163A | rs622106 |

| | | Greece 2 | Greece 3 | Greece 4 | Greece 5 | Greece 6 | Greece 7 |
|---|---|---|---|---|---|---|---|
| | Greece 1 | (the 1^{st} | (the 2^{nd} | (the 3rd | (the 1^{st} | (the 2^{nd} | (the 3^{rd} |
| Chromosome | (grandmother) | daughter) | daughter) | daughter) | granddaughter) | granddaughter) | granddaughter) |
| 4 | ./. | 1/1 | 0/1 | 1/1 | 1/1 | 1/1 | 1/1 |
| 4 | 0/1 | 1/1 | ./. | 1/1 | 1/1 | 1/1 | 1/1 |
| 4 | 0/1 | ./. | 0/1 | ./. | ./. | ./. | ./. |
| 4 | ./. | ./. | ./. | ./. | 0/1 | 0/1 | ./. |
| 4 | 1/1 | ./. | 1/1 | ./. | ./. | ./. | 0/1 |
| 4 | 1/1 | ./. | 0/1 | ./. | ./. | ./. | ./. |
| 4 | ./. | ./. | 0/1 | ./. | ./. | ./. | ./. |
| 4 | | | | | | | |
| 4 | 1/1 | ./. | ./. | ./. | ./. | ./. | ./. |
| 4 | 1/1 | ./. | 0/1 | ./. | ./. | ./. | ./. |
| 4 | 1/1 | ./. | 0/1 | ./. | ./. | ./. | ./. |
| 4 | ./. | ./. | 0/1 | ./. | ./. | ./. | 0/1 |
| 4 | ./. | ./. | 0/1 | ./. | ./. | ./. | ./. |
| 4 | ./. | ./. | 0/1 | ./. | ./. | ./. | 0/1 |
| 4 | 1/1 | 1/1 | 1/1 | 1/1 | 1/1 | 1/1 | 0/1 |
| 8 | 0/1 | 0/1 | 0/1 | 0/1 | 0/1 | 0/1 | 1/1 |
| 8 | 0/1 | 0/1 | 1/1 | 1/1 | 0/1 | 0/1 | 0/1 |
| 8 | ./. | 0/1 | 0/1 | 0/1 | ./. | ./. | 0/1 |
| 8 | 1/1 | ./. | ./. | ./. | ./. | ./. | ./. |
| 8 | 1/1 | ./. | ./. | ./. | 1/1 | 1/1 | ./. |
| 8 | ./. | ./. | 1/1 | 1/1 | ./. | ./. | 0/1 |
| 8 | ./. | ./. | 0/1 | 0/1 | ./. | ./. | ./. |
| 8 | 1/1 | 1/1 | 1/1 | 1/1 | 1/1 | 1/1 | 1/1 |
| 8 | 0/1 | 0/1 | 0/1 | 0/1 | ./. | ./. | 1/1 |

TABLE 2
Additional variants associated with endometriosis

| | | REF | Minor | | Variant | Nucle | AA | | |
|---|---|---|---|---|---|---|---|---|---|
| Chromosome | Position | Allele | Allele | GENE | type | change | Change | transcript | dbsnp |
| chr1 | 170136876 | T | C | METTL11B | Missense | c.T830C | p.L277P | NM_001 | rs144066772 |
| | | | | | | | | 136107 | |
| chr1 | 170129701 | T | C | METTL11B | Missense | c.T197C | p.M66T | NM_001 | rs147253400 |
| | | | | | | | | 136107 | |
| chr1 | 170115300 | G | C | METTL11B | Missense | c.G52C | p.D18H | NM_001 | rs138142057 |
| | | | | | | | | 136107 | |

| | gnomAD freq | | | | | gnomAD freq | |
|---|---|---|---|---|---|---|---|
| Chromosome | (all populations) | P | OR | L95 | U95 | (non-Finnish Europeans only) | EndoFreq |
| chr1 | 0.0083 | 1 | 0.99 | 0.73 | 1.35 | 0.0109 | 0.0108 |
| chr1 | 0.0055 | 0.330993 | 1.32 | 0.76 | 2.29 | 0.0068 | 0.0090 |
| chr1 | 0.0009 | 1 | 1.00 | 0.31 | 3.22 | 0.0007 | 0.0007 |

While exemplary embodiments of the present disclosure have been shown and described herein, it will be apparent to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all embodiments of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

1.-103. (canceled)

104. A method comprising:
detecting a presence or an absence of a genetic variant in genetic material from a human subject suspected of having or developing endometriosis, wherein the genetic variant is selected from Table 1 or Table 2.

105. The method of claim 104, wherein the genetic variant defines a minor allele.

106. The method of claim 104, wherein the genetic variant comprises a synonymous mutation, a non-synonymous mutation, a nonsense mutation, an insertion, a deletion, a splice-site variant, a frameshift mutation, a protein damaging mutation, or any combination thereof.

107. The method of claim 104, wherein the genetic variant is of a gene selected from the group consisting of UGT2B28, USP17L2, METTL11B, and any combination thereof.

108. The method of claim 107, wherein the genetic variant is of UGT2B28.

109. The method of claim 107, wherein the genetic variant is of USP17L2.

110. The method of claim 107, wherein the genetic variant is of METTL11B.

111. The method of claim 104, wherein the genetic material comprises mRNA, cDNA, genomic DNA, PCR amplified products produced therefrom, or any combination thereof.

112. The method of claim 104, wherein the genetic material is at least partially isolated from a blood sample.

113. The method of claim 104, wherein the genetic material comprises cell-free DNA.

114. The method of claim 104, wherein the detecting comprises sequencing at least a portion of the genetic material; hybridizing a probe complementary to a portion of the genetic material; labeling the genetic variant; performing an oligonucleotide ligation assay; performing a PCR-based assay; or any combination thereof.

115. The method of claim 114, wherein the detecting comprises the hybridizing, and wherein the probe complementary to the portion of the genetic material is a sequencing primer or an allele specific probe.

116. The method of claim 114, wherein the detecting comprises the labeling, and wherein the genetic variant is labeled with a fluorescent label.

117. The method of claim 104, wherein the detecting yields a data set.

118. The method of claim 117, further comprising inputting the data set into a programmed computer having a trained algorithm.

119. The method of claim 118, further comprising outputting an electronic report that comprises a result of the detecting.

120. The method of claim 104, further comprising administering a therapeutic to the human subject.

121. The method of claim 120, wherein the therapeutic comprises a regenerative therapy, a medical device, a pharmaceutical composition, a medical procedure, or any combination thereof.

122. The method of claim 104, wherein the human subject is asymptomatic for endometriosis.
